(19) United States
(12) Patent Application Publication
(10) Pub. No.: US 2011/0212163 A1
(43) Pub. Date: Sep. 1, 2011

(54) MAGNETIC HEATING FOR DRUG DELIVERY AND OTHER APPLICATIONS

(75) Inventors: Todd R. Hoare, Ontario (CA); Daniel S. Kohane, Newton, MA (US); Robert S. Langer, Newton, MA (US); Mikhail G. Shapiro, Boston, MA (US); Yoon Yeo, Cambridge, MA (US); Grace Y. Kim, Cambridge, MA (US); Shahriar R. Khushrushahi, Cambridge, MA (US)

(73) Assignees: CHILDREN'S MEDICAL CENTER CORPORATION, Boston, MA (US); MASSACHUSETTS INSTITUTE OF TECHNOLOGY, Cambridge, MA (US); THE GENERAL HOSPITAL CORPORATION D/B/A, Boston, MA (US)

(21) Appl. No.: 13/055,195

(22) PCT Filed: Jul. 23, 2009
(86) PCT No.: PCT/US2009/004268

§ 371 Date: May 23, 2011

Related U.S. Application Data
(60) Provisional application No. 61/083,458, filed on Jul. 24, 2008.
(60) Provisional application No. 61/166,526, filed on Apr. 3, 2009.

(51) Int. Cl. A61K 47/02; A61K 9/127

(52) U.S. Cl. 424/450; 514/769

(57) ABSTRACT

The present invention generally relates to systems and methods for releasing a compound from an article using an external trigger, for example, magnetic fields. One aspect of the invention is generally directed to an article containing a magnetically-susceptible material. Exposure of the magnetically-susceptible material to a magnetic field, such as an oscillating magnetic field, may cause the magnetically-susceptible material to increase in temperature. This increase in temperature may be used, in some embodiments, to cause the release of a drug or other releasable material from the article. For instance, the drug may be contained in a heat-sensitive material in thermal communication with the magnetically-susceptible material, or the drug may be contained within an enclosure that is isolated, at least in part, by a heat-sensitive material in thermal communication with the magnetically-susceptible material. Other aspects of the invention are directed to systems and methods of making or using such articles, e.g., by implanting the article within a subject, methods of treatment involving such articles, kits including such articles, or the like.

### RELATED APPLICATIONS

This application claims the benefit of U.S. Provisional Patent Application Ser. No. 61/166,526, filed Apr. 3, 2009, entitled “Magnetic Heating for Drug Delivery and Other Applications,” by Hoare, et al.; and to U.S. Provisional Patent Application Ser. No. 61/083,458, filed Jul. 24, 2008, entitled “Externally-Triggered Thermosensitive Membranes,” by Hoare, et al. Each of these is incorporated herein by reference.

### GOVERNMENT FUNDING

Research leading to various aspects of the present invention were sponsored, at least in part, by the National Institutes of Health, Grant No. GM 073626. The U.S. Government has certain rights in the invention.

### FIELD OF INVENTION

The present invention generally relates to systems and methods for releasing a releasable species from an article using an external trigger, for example, using magnetic fields.

### BACKGROUND

Controlled-release and sustained-release techniques for delivering drugs to a subject have been well-studied. Such techniques generally involve the use of delivery vehicles such as pills, tablets, capsules, implants, and the like that are formulated to dissolve slowly and release a drug over time. However, in such techniques, the delivery profile for the drug must be “pre-programmed” within the delivery vehicle itself. For example, an implant may be engineered to release a drug at a predetermined rate once the implant has been implanted within a subject. If, however, the medical condition of the subject changes or the drugs or the dosage of the drug needs to be altered in some way, e.g., reduced, increased, eliminated, etc., the implant itself within the subject must be somehow altered, for example, removed via surgery and replaced with another implant engineered to release the drug (or a new drug) at a new, predetermined rate. This involves considerable time, expense, and potential risk to the subject.

While some devices have been developed that allow for externally-controlled release of drugs, such devices typically are based on silicon circuitry or other electronic devices that are implanted into a subject, and are often powered by a battery. Such devices are typically not fully biologically compatible, and physiological conditions (liquid, cells, etc.) often create problems with the electronic circuitry of the device. Thus, further advances are needed.

### SUMMARY OF THE INVENTION

The present invention generally relates to systems and methods for releasing a compound from an article using an external trigger, for example, using magnetic fields. The subject matter of the present invention involves, in some cases, interrelated products, alternative solutions to a particular problem, and/or a plurality of different uses of one or more systems and/or articles.

One aspect of the invention is directed to an article. In one set of embodiments, the article contains a magnetically-susceptible material at least partially defining an enclosure containing a releasable species. In one embodiment, application of an oscillating magnetic field to the magnetically-susceptible material causes at least some release of the releasable species externally from the enclosure. In another embodiment, application of an oscillating magnetic field to the magnetically-susceptible material causes an increase of release of the releasable species of at least about 10% from the implantable article, relative to the amount of release of the releasable species from the article in the absence of oscillating magnetic field. In yet another embodiment, the magnetically-susceptible material is in thermal communication with a heat-sensitive material, where application of an oscillating magnetic field to the magnetically-susceptible material causes the heat-sensitive material to increase in temperature by at least about 0.5° C.

In one set of embodiments, the article includes a membrane having a first permeability when an oscillating magnetic field is applied to the membrane, and a second permeability in the absence of the oscillating magnetic field. The article, in another set of embodiments, includes a membrane having a first permeability when the membrane is at a temperature of less than about 37° C. and a second permeability when the membrane is at a temperature of greater than about 37° C., the second permeability being at least 50% greater than the first permeability.

The invention, in another aspect, is directed to a method. According to a first set of embodiments, the method includes an act of applying an oscillating magnetic field to at least a portion of an article defining an enclosure containing a releasable species, where the article contains a magnetically-susceptible material at least partially defining the enclosure, to cause an increase of at least about 10% in the release of the releasable species from the article, relative to the amount of release of the releasable species from the article in the absence of the oscillating magnetic field.

The method, in another set of embodiments, includes acts of applying an oscillating magnetic field to at least a portion of an article defining an enclosure containing a releasable species, where the article contains a magnetically-susceptible material at least partially defining the enclosure, to cause an increase in temperature of at least about 0.5° C. of the magnetically-susceptible material. In yet another set of embodiments, the method includes an act of implanting an article implanted internally within a subject, where the article contains a magnetically-susceptible material at least partially defining an enclosure containing a releasable species. In some cases, application of an oscillating magnetic field to the magnetically-susceptible material causes an increase of at least about 10% in the release of the releasable species from the article, relative to the amount of release of the releasable species from the article in the absence of the oscillating magnetic field.

In one set of embodiments, the method includes an act of reversibly altering the permeability of a membrane by applying an oscillating magnetic field to at least a portion of the membrane.

The method, in yet another set of embodiments, is a method of treating cancer. In one set of embodiments, the method includes an act of directing an oscillating magnetic field at tissue suspected of being cancerous, where the tissue contains an implanted article containing an anti-cancer drug, to cause an increase of at least about 10% in the release of the drug from the article, relative to the amount of release of the drug from the material in the absence of the oscillating magnetic field.

According to still another set of embodiments, the method is a method for administering a drug to a subject having a chronic disease. The method, in one embodiment, includes an act of directing an oscillating magnetic field at an implanted article containing a drug for treating the chronic disease to cause an increase of at least about 10% in the release of the drug from the article, relative to the amount of release of the drug from the material in the absence of the oscillating magnetic field. In some cases, the chronic disease is not cancer.

Yet another set of embodiments of the present invention is directed to a method for administering anesthesia at a site in a subject in need thereof. In one embodiment, the method includes acts of administering to a subject at a site at which anesthesia is desired, an implanted article comprising an effective amount of a anesthetic, and directing an oscillating magnetic field at the article in an amount effective to release the local anesthetic.

In another aspect, the present invention is directed to a method of making one or more of the embodiments described herein, for example, an article comprising a magnetically-susceptible material. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic diagram illustrating activation of an article of the invention using electromagnetic radiation;

FIG. 2 illustrates an article prepared according to one embodiment of the invention;

FIG. 3 illustrates the release of a tracer, in another embodiment of the invention;

FIG. 4 illustrates the release of a tracer in various articles, in other embodiments of the invention;

FIG. 5 illustrates the release of a tracer from an article, in yet another embodiment of the invention;

FIG. 6 illustrates the release of tracers from various articles, in certain embodiments of the invention;

FIG. 7 illustrates the release of a tracer from certain articles, in other embodiments of the invention;

FIG. 8 illustrates the release of a tracer from an article in one embodiment of the invention;

FIG. 9 illustrates the release of a tracer from an article in another embodiment of the invention;

FIG. 10 illustrates the release of a drug from various articles produced in other embodiments of the invention;

FIG. 11 illustrates biocompatibility assays of articles of certain embodiments of the invention;

FIGS. 12A-12B are photographs illustrating the biocompatibility of certain embodiments of the invention;

FIG. 13 illustrates the release of a tracer from various implanted articles, in certain embodiments of the invention;

FIG. 14 illustrates the temperature dependence of the flux of a tracer, in yet another embodiment of the invention;

FIG. 15 illustrates gel particle size as a function of temperature, in yet another embodiment of the invention;

FIGS. 16A-16B illustrate magnetically-triggered release of a tracer according to another embodiment of the invention; and

FIGS. 17A-17C illustrate magnetically-triggered release of a tracer according to yet another embodiment of the invention.

### DETAILED DESCRIPTION

The present invention generally relates to systems and methods for releasing a compound from an article using an external trigger, for example, magnetic fields. One aspect of the invention is generally directed to an article containing a magnetically-susceptible material. Exposure of the magnetically-susceptible material to a magnetic field, such as an oscillating magnetic field, may cause the magnetically-susceptible material to increase in temperature. This increase in temperature may be used, in some embodiments, to cause the release of a drug or other releasable material from the article. For instance, the drug may be contained in a heat-sensitive material in thermal communication with the magnetically-susceptible material, or the drug may be contained within an enclosure that is isolated, at least in part, by a heat-sensitive material in thermal communication with the magnetically-susceptible material. Other aspects of the invention are directed to systems and methods of making or using such articles, e.g., by implanting the article within a subject, methods of treatment involving such articles, kits including such articles, or the like.

One aspect of the present invention is generally directed to articles containing a magnetically-susceptible material and a releasable species (such as a drug) that can be released from the article, typically upon application of a magnetic field, such as an oscillating magnetic field, to the magnetically-susceptible material, or at least a portion of it. For instance, in some cases, the article is one that releases the releasable species at a first rate in the absence of a magnetic field, but at a second rate when a magnetic field is applied, i.e., application of the magnetic field to the article can be used to increase or decrease the rate of release of the releasable species from the article. The releasable species may be released, for example, due to heating of the magnetically-susceptible material, and/or other effects, for example, mechanical shaking or oscillation of the magnetically-susceptible material to cause release to occur.

As a non-limiting example, in one set of embodiments, an article containing a magnetically-susceptible material may be heated by directing a magnetic field at at least a portion of the article. The magnetically-susceptible material may be, for example, iron, or a ferrofluid. The magnetically-susceptible material can be positioned to be in thermal communication with a heat-sensitive material, such as poly(N-isopropylacrylamide). A releasable species, such as a drug, may be released upon heating of the heat-sensitive material, or upon cooling the heat-sensitive material in some cases. For instance, the heat-sensitive material may contain pores containing the releasable species, and as the heat-sensitive material is heated, the pores open, allowing more of the releasable species to be released. Accordingly, a magnetic field can be directed at the magnetically-susceptible material (or portion thereof) to heat the magnetically-susceptible material, which in turn heats the heat-sensitive material, causing a releasable species to be released from the article (or causing a change in the rate of release of the releasable species from the article).

Typically, a magnetic field is directed at a magnetically-susceptible material to heat the material, or otherwise alter the material. As used herein, a “magnetically-susceptible material” is one which experiences magnetism (permanent or temporarily) in response to an applied magnetic field. Such materials may be readily identified using simple screening tests to measure the magnetic susceptibility or the relative permeability of the material. For instance, a magnetic field of about 2 mT may be applied to a sample of the material, and the magnetic susceptibility or the relative permeability of the material may be determined under such conditions. Often, a magnetically-susceptible material will be one which exhibits some degree of magnetization under such conditions. For instance, the magnetically-susceptible material may be one which exhibits a magnetic susceptibility (Xₘ) of at least about 10⁻⁵ or at least about 10⁻⁴. Non-limiting examples of magnetically-susceptible materials include iron, nickel, cobalt, gadolinium, alloys of these metals with each other and/or with other metals, rare earth metals, certain ceramics, ferrofluids, or the like. Typically, as is known to those of ordinary skill in the art, a ferrofluid contains magnetically susceptible particles contained within a fluid.

In some cases, the magnetically-susceptible material is a material that can be heated by at least about 0.5° C. by directing a magnetic field at the material, for example, an alternating or oscillating magnetic field. For example, the material may be heated by at least about 0.5° C., at least about 1° C., at least about 2° C., at least about 4° C., at least about 5° C., or more in some cases, for instance any integer up to and including 20° C., depending on factors such as the intensity and/or frequency of the magnetic field, the magnetic susceptibility of the magnetically-sensitive material at those frequencies, any intervening materials between the source of magnetism and the magnetically-susceptible material, or the like. Heating of the material may be caused, for example, due to eddy currents within the material. Heating of the magnetically-susceptible material may be used to heat other materials, such as a heat-sensitive material positioned in thermal communication with the magnetically-susceptible material.

The magnetic field may arise from any suitable source of magnetism, for example, using a permanent magnet, a temporary magnet such as an electromagnet, or the like. Many of these sources are available commercially. Thus, for example, the oscillating magnetic field may be one that varies between positive and negative values, or one that varies between two different values having the same sign. The change in the magnetic field over time (i.e., its waveform) may be of any suitable waveform, i.e., a sine wave, a square wave, a triangular wave, or the like. Typically, the field strength of the applied magnetic field may be at least about 10⁻⁵ T or at least about 10⁻⁴ T, or greater in some cases. If an oscillating magnetic field is used, the frequency of oscillations may be any suitable frequency. In some cases, relatively high frequencies may be desirable to allow for faster heating of the magnetically-susceptible material. For instance, the frequency of the oscillations may be at least about 1 kHz, at least about 10 kHz, at least about 100 kHz, at least about 1000 kHz, or more in some cases. For example, in one embodiment, a magnetic field of 260 kHz at 0-20 mT may be useful. Without wishing to be bound by any theory, it is believed that the relatively fast switching of the polarity and/or the magnetic field strength results in the transfer of magnetic energy to the implant, which can then be expressed as heat. The heat may be used to, for example, heat a heat-sensitive material that my be positioned in thermal communication with the magnetically-susceptible material.

As used herein, a “heat-sensitive material” is a material that alters its size (linearly) by at least about 0.004% in response to a change in temperature by at least about 0.5° C. or at least 1° C. The heat-sensitive material may increase or decrease in size, depending on the type of material. In some cases, the alteration may be at least about 0.01%, at least about 0.03%, at least about 0.1%, at least about 0.3%, or at least about 1%, and in some cases, this change is measured under physiologically-relevant conditions (e.g., at a temperature of 37° C.). For example, in some cases, a size change may result from a change in the affinity of the polymers for water as the temperature increases, causing the absorption of expulsion of water from the polymer network. In some embodiments, the radiation sensitive material may be the same as the heat sensitive material.

The heat-sensitive material may be a polymer in some cases. Examples of heat-sensitive polymers include, but are not limited to, poly(N-isopropylacrylamide) or other poly(N-alkyacrylamide)s or poly(N-alkylmethacrylamide)s such as poly(N-ethylacrylamide), poly(N-t-butylacrylamide), poly(N-methylacrylamide), poly(N-isopropylmethacrylamide), etc. Other examples of heat-sensitive polymers include poloxamer 407, poloxamer 188, Pluronic® F127, Pluronic® F68, poly(methyl vinyl ether), poly(N-vinylcaprolactam), or poly(organophosphazenes). It should also be understood that other components may be used to alter the sensitivity of the heat-sensitive polymers to changes in temperature, for instance, added as a copolymer component, and/or as a separate component. Examples include (but are not limited to) acrylic acid, methacrylic acid, N-vinylpyrrolidone, N,N-dimethyl aminoethylmethacrylate, oxazoline, butylmethacrylate, acrylamide, or any other vinyl or acrylic monomer which can be copolymerized with the thermosensitive monomers. Block copolymers comprising one or more hydrophilic block and/or one or more hydrophobic block may also be used in some cases. For example, block copolymers of poly(ethylene glycol) with polylactide, polyglycolide, poly(lactide-co-glycolide) (PLGA), or poly(methyl methacrylate) may be used. In some cases, the heat-sensitive polymer may be present with other polymers, for example, polymers for providing a structural matrix. Examples of such polymers include, but are not limited to, poly(ethylene glycol), polylactide, polyglycolide, poly(methyl methacrylate), or the like. For instance, the two polymers may be present as a polymer blend, a co-polymer, or as interpenetrating polymers.

As used herein, an “interpenetrating polymer network” or an “IPN” is a polymeric material comprising two or more networks of two or more polymers (including copolymers) which are at least partially interlaced on a molecular scale, but not covalently bonded to each other and cannot be separated, even theoretically, unless chemical bonds are broken. Thus, a mixture of two or more pre-formed polymer networks (e.g., a mixture or a blend) is not an interpenetrating polymer network. Specific non-limiting examples of an interpenetrating network include [net-poly(styrene-stat-butadiene)]-ipn-[net-poly(ethyl acrylate)]. Those of ordinary skill in the art are able to form IPNs, for example, by blending different polymer precursors which have the ability under set conditions to react to form two or more different interpenetrating polymers that do not bind to each other, by forming a first polymer and allowing a precursor of a second polymer to diffuse into the first polymer in an interpenetrating manner and to react to form the second polymer under conditions that do not promote binding between the first and second polymer, by blending two or more linear or branched polymers with at least one polymer having pendant reactant groups and subsequently adding a chain extender to cross-link each of the polymers into separate networks, and/or by proceeding with a multi-stage polymerization process including a first polymer network that is partially polymerized to allow for high swellability and/or easy diffusion of a second polymer precursor, allowing the second polymer precursor to penetrate the first polymer network, and thereafter polymerizing both polymer networks, etc.

As mentioned, the heat-sensitive material may be positioned to be in thermal communication with the magnetically susceptible material, i.e., such that an increase in temperature of the magnetically susceptible material results in an increase in the temperature of the heat-sensitive material. Thus, heat produced by the magnetically susceptible material, upon exposure to a magnetic field, may be transferred to the heat-sensitive material. The transfer of heat may be direct (e.g., if the magnetically susceptible material and the heat-sensitive material are positioned in direct physical contact, or if the magnetically susceptible material and the heat-sensitive material are mixed together), or indirect (e.g., one or more intervening materials are used to transfer heat from the magnetically susceptible material to the heat-sensitive material). Both examples are encompassed by “positioned in thermal communication.” In some cases, such intervening materials may have relatively high thermal conductivities, for example, at least about 100 to about 400 W/m K. For instance, an intervening material may comprise a metal, such as aluminum, copper, gold, silver, and the like. It should also be noted that in some cases, such materials may also be used for other purposes within the article.

The heat-sensitive material, upon heating, may cause or stop the release, or otherwise cause a change in the release rate, of a drug or other releasable species from the article. For example, the article may begin releasing a releasable species, or stop the release of a releasable species, or the article may exhibit a change in the rate of release of the releasable species from the article. As non-limiting examples, the article may exhibit an increase of at least about 5%, at least about 10%, at least about 15%, at least about 20%, at least about 30%, at least about 40%, at least about 50%, at least about 75%, at least about 100%, at least about 300%, at least about 500%, at least about 1000%, at least about 2000%, at least about 3000%, etc., in the release of releasable species from the article, relative to the amount of release of the releasable species from the article in the absence of a magnetic field. Heating of the heat-sensitive material to cause or stop the release, or cause a change in the release rate, may be caused by applying a magnetic field to the magnetically susceptible material, as previously discussed, and/or other methods may be used to heat the heat-sensitive material. For example, the heat-sensitive material may be heated by applying heat from a heat source to the article, or a portion thereof, for instance, to the heat-sensitive material, to an intervening material between the heat-sensitive material and the magnetically susceptible material, or the like. Such applications may be useful, for instance, to further control release of the releasable species from the article, e.g., in addition to the magnetic field.

The releasable species may be at least partially contained within the heat-sensitive material, and/or contained within an enclosure. For instance, the enclosure may be isolated, at least in part, by the heat-sensitive material. In one embodiment, the transport of the releasable species from the enclosure across the heat-sensitive material is altered upon heating of the heat-sensitive material. For example, the diffusion coefficient of the releasable species across the heat-sensitive material may be altered upon heating. In another embodiment, the heat-sensitive material may be used to fill the pores of the membrane such that the temperature of the heat-sensitive material can be used to change the average free volume of the pores within the membrane. In such embodiments, the releasable species may be contained within the pores themselves and/or within an enclosure of the article such that the drug can be transported through the pores (e.g., via diffusion through the pores) for release.

In one set of embodiments, the heat-sensitive material comprises a gel, and the releasable species may be contained within the gel, e.g., within the porous polymeric network of the gel. For example, the heat-sensitive material may contain heat-sensitive polymers such as poly(N-isopropylacrylamide), or other polymers discussed above. In some cases, the temperature at which the heat-sensitive polymeric gel swells can be tuned by copolymerizing a heat-sensitive polymer with other monomers. For instance, comonomers having different hydrophilicities compared to the heat-sensitive polymer can be used to tune the transition temperature; for example, more hydrophilic comonomers result in higher transition temperatures while more hydrophobic comonomers result in lower transition temperatures. In other cases, comonomers with stiffer backbones (i.e., methacrylamide-based monomers) can be used to increase the phase transition temperature of the heat-sensitive polymer, e.g., by restricting the mobility of the hydrophobic segments to aggregate as the temperature increases. An example of this behavior is discussed in the examples, below.

As discussed, the heat-sensitive material may be used to control release of a drug or other releasable species from the article. The drug or other releasable species may be present within the article in any form, e.g., as a solid, as a liquid, contained within an aqueous or an organic solution, or the like. In one set of embodiments, the drug or other releasable species may be present as a controlled release formulation that can release drug over an extended period of time (e.g., at least over 24 hours, and often over a week or more, even when exposed to a pure water environment). The releasable species may be contained within an enclosure (if one is present), and/or contained within the heat-sensitive material, e.g., as a component of the heat-sensitive material and/or contained within pores within the heat-sensitive material. In one set of embodiments, the releasable species may is a drug or other compound where the control of release from the article is desired. For example, the drug may be a small molecule (e.g., having a molecular weight of less than about 1000 Da), a protein or a peptide, a nucleic acid, a hormone, a vitamin, or the like. In some cases, the releasable species may be present as particles, such as nanoparticles. For example, the particles may have an average diameter of less than about 1 micrometer, less than about 500 nm, less than about 400 nm, less than about 300 nm, less than about 100 nm, less than about 50 nm, less than about 30 nm, less than about 10 nm, etc.

The article, in some embodiments, at least partially defines an enclosure containing the releasable species. An enclosure is a space, filled wholly or partially, bounded by material, such as the heat sensitive material. The heat sensitive material, for instance, may form the enclosure and separate the releasable species from the magnetically-susceptible polymer. The enclosure may be, for example, a physical device (e.g., an impermeable container having an opening that can release the releasable species controlled by the heat sensitive material), or in some cases, the enclosure may be a particle or a vesicle such as a liposome formed by or including the heat sensitive material. The enclosure may contain some or all of the releasable species within the article. The releasable species can be present in the enclosure in any form, for instance, as a solid, in an aqueous solution, or in a controlled release formulation. An “aqueous solution,” as used herein, is one which is miscible in pure water. Examples include, but are not limited to, ethanol, water containing a salt, a surfactant, or an emulsifier, or pure water itself.

In some cases, there may be more than one heat-sensitive material present within the article and/or more than one magnetically-susceptible material present within the article. In some cases, such materials may be used for multiplex control of the article, e.g., a first magnetic field may be used to preferentially interact with a first magnetically-susceptible material while a second magnetic field (e.g., at a different frequency or intensity) may be used to preferentially interact with a second magnetically-susceptible material. For example, in one embodiment, the article may have a first enclosure and a second enclosure, and different magnetic fields may be used to cause release from the first enclosure or the second enclosure, e.g., of the same or different releasable species.

In some cases, the articles may be used in non-medical and industrial applications such as bioseparation, filtration, medical diagnostics, or the like. For instance, in one set of embodiments, an article may be used to control a bioseparation process. A magnetically-susceptible polymer (or other material), and a heat-sensitive material may be used to form a membrane. The membrane may be, for example, attached to a physical device, or formed into a microparticle, a sphere comprising polymers, etc. The membrane may be such that the permeability and/or selectivity of the membrane, for example, for specific biomolecules, may be dynamically controlled. Thus, for example, the membrane may exhibit a first permeability or selectivity in the absence of a magnetic field, and a second permeability or selectivity when a magnetic field is applied. In some; cases, multiple permeabilities or selectivities may be exhibited by the membrane, e.g., by the application of different magnetic field intensities, or frequencies of magnetic fields (e.g., in the case of an oscillating magnetic field). In addition, in some embodiments, the permeability or selectivity may be repeatedly altered, e.g., between these states. The membrane, in one embodiment, may have a first permeability at a temperature below a certain transition temperature and a second permeability at a temperature above the transition temperature. As a non-limiting example, the transition temperature may be about 37° C., such that the membrane exhibits a first permeability to a species when implanted in a subject, but that the membrane can be switched to a second permeability by heating the membrane in some fashion, e.g., by applying oscillating magnetic field to a magnetically-susceptible material in thermal communication with the membrane.

As another example, an article may be used to control access by a sensor, e.g., a sensor contained within the enclosure. The enclosure may be isolated, at least in part, by a heat-sensitive material positioned in thermal communication with the magnetically-susceptible material. Access to the enclosure may be controlled by the heat-sensitive material such that the heat-sensitive material exhibits a first permeability or selectivity to an analyte in the absence of a magnetic field and a second permeability or selectivity to the analyte when a magnetic field is applied. Thus, the sensor may be activated for sensing, or protected when not in use, by the application of the magnetic field. Such a sensor may be used in numerous applications, for example within an industrial process, as an implant within a subject, or the like.

In yet another example, such an article may be useful for environmentally-sensitive packaging. For instance, a dye could be contained within an enclosure, and released when certain conditions are met or exceeded, for instance, when the article reaches a certain temperature, or when the article is exposed to a magnetic field. Detection of the dye would then be useful for determining whether the article has been exposed to certain environmental stimuli.

As another example, the article may be used for the controlled release of a drug or other releasable species to a subject. The term “controlled release” generally refers to compositions, e.g., pharmaceutically acceptable carriers, for controlling the release of an active agent or drug incorporated therein, typically by slowing the release of the active agent or drug in order to prevent immediate release. Such controlled release compositions and/or carriers can be used herein to prolong or sustain the release of an active agent or drug incorporated, e.g., a chemotherapeutic or an anesthetic. Thus, the terms “controlled release” and “sustained release” are generally used interchangeably throughout this document unless otherwise indicated.

The releasable species may be a drug such as a therapeutic, diagnostic, or prophylactic agent. Releasable species include, for instance, small molecules, organometallic compounds, nucleic acids (e.g., DNA, RNA, RNAi, etc.), proteins, peptides, metals, an isotopically labeled chemical compounds, vaccines, immunological agents, etc.

In one embodiment, the releasable species are organic compounds with pharmaceutical activity, such as, for instance, a clinically used drug. Examples of releasable species include an antibiotic, anti-viral agent, anesthetic, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, non-steroidal anti-inflammatory agent, nutritional agent, etc. The drug may be used to treat any condition, such as cancer (e.g., as a chemotherapeutic agent), a chronic disease (not necessarily cancer, e.g., epilepsy, a neurodegenerative disease, a cardiovascular disease, an autoimmune disease, diabetes, etc.), etc. In one embodiment, the drug is an anesthetic, such as an amino amide anesthetic selected from the group comprising bupivacaine, levobupivacaine, lidocaine, mepivacaine, ropivacaine, tetracaine, prilocaine, ropivacaine, articaine, trimecaine and their salts and prodrugs. Other non-limiting examples of anesthetics include tetrodotoxin, saxitoxin, or similar compounds (e.g., site 1 sodium channel blockers).

Further non-limiting examples of drugs or other releasable species that may be used include antimicrobial agents, analgesics, antinflammatory agents, counterirritants, coagulation modifying agents, diuretics, sympathomimetics, anorexics, antacids and other gastrointestinal agents; antiparasitics, antidepressants, antihypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, para-sympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anticancer properties, or combinations thereof. Additional therapeutic agents which may be administered in accordance with the present invention include, without limitation: antiinfectives such as antibiotics and antiviral agents; analgesics and analgesic combinations; anorexics; antiheimintics; antiarthritics; antiasthmatic agents; anticonvulsants; antidepressants; antidiuretic agents; antidiarrleals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Specific non-limiting examples include acebutolol, acetaminophen, acetohydoxamic acid, acetophenazine, acyclovir, adrenocorticoids, allopurinol, alprazolam, aluminum hydroxide, amantadine, ambenonium, amiloride, aminobenzoate potassium, amobarbital, amoxicillin, amphetamine, ampicillin, androgens, anesthetics, anticoagulants, anticonvulsants-dione type, antithyroid medicine, appetite suppressants, aspirin, atenolol, atropine, azatadine, bacampicillin, baclofen, beclomethasone, belladonna, bendroflumethiazide, benzoyl peroxide, benzthiazide, benztropine, betamethasone, betha nechol, biperiden, bisacodyl, bromocriptine, bromodiphenhydramine, brompheniramine, buclizine, bumetanide, busulfan, butabarbital, butaperazine, caffeine, calcium carbonate, captopril, carbamazepine, carbenicillin, carbidopa & levodopa, carbinoxamine inhibitors, carbonic anhydsase, carisoprodol, carphenazine, cascara, cefaclor, cefadroxil, cephalexin, cephradine, chlophedianol, chloral hydrate, chlorambucil, chloramphenicol, chlordiazepoxide, chloroquine, chlorothiazide, chlorotrianisene, chlorpheniramine, chlorpromazine, chlorpropamide, chlorprothixene, chlorthalidone, chlorzoxazone, cholestyramine, cimetidine, cinoxacin, clemastine, clidinium, clindamycin, clofibrate, clomiphere, clonidine, clorazepate, cloxacillin, colochicine, coloestipol, conjugated estrogen, contraceptives, cortisone, cromolyn, cyclacillin, cyclandelate, cyclizine, cyclobenzaprine, cyclophosphamide, cyclothiazide, cycrimine, cyproheptadine, danazol, danthron, dantrolene, dapsone, dextroamphetamine, dexamethasone, dexchlorpheniramine, dextromethorphan, diazepan, dicloxacillin, dicyclomine, diethylstilbestrol, diflunisal, digitalis, diltiazen, dimenhydrinate, dimethindene, diphenhydramine, diphenidol, diphenoxylate & atrophive, diphenylopyraline, dipyradamole, disopyramide, disulfiram, divalporex, docusate calcium, docusate potassium, docusate sodium, doxorubicin, doxyloamine, dronabinol ephedrine, epinephrine, epirubicin, ergoloidmesylates, ergonovine, ergotamine, erythromycins, esterified estrogens, estradiol, estrogen, estrone, estropipute, etharynic acid, ethchlorvynol, ethinyl estradiol, ethopropazine, ethosaximide, ethotoin, fenoprofen, ferrous fumarate, ferrous gluconate, ferrous sulfate, flavoxate, flecainide, fluphenazine, fluprednisolone, flurazepam, folic acid, furosemide, gemfibrozil, glipizide, glyburide, glycopyrrolate, gold compounds, griseofiwin, guaifenesin, guanabenz, guanadrel, guanethidine, halazepam, haloperidol, hetacillin, hexobarbital, hydralazine, hydrochlorothiazide, hydrocortisone (cortisol), hydroflunethiazide, hydroxychloroquine, hydroxyzine, hyoscyamine, ibuprofen, indapamide, indomethacin, insulin, iofoquinol, iron-polysaccharide, isoetharine, isoniazid, isopropamide isoproterenol, isotretinoin, isoxsuprine, kaolin, pectin, ketoconazole, lactulose, levodopa, lincomycin liothyronine, liotrix, lithium, loperamide, lorazepam, magnesium hydroxide, magnesium sulfate, magnesium trisilicate, maprotiline, meclizine, meclofenamate, medroxyproyesterone, melenamic acid, melphalan, mephenytoin, mephobarbital, meprobamate, mercaptopurine, mesoridazine, metaproterenol, metaxalone, methamphetamine, methaqualone, metharbital, methenamine, methicillin, methocarbamol, methotrexate, methsuximide, methyclothinzide, methylcellulos, methyidopa, methylergonovine, methylphenidate, methylprednisolone, methysergide, metoclopramide, matolazone, metoprolol, metronidazole, minoxidil, mitotane, monamine oxidase inhibitors, nadolol, nafcillin, nalidixic acid, naproxen, narcotic analgesics, neomycin, neostigmine, niacin, nicotine, nifedipine, nitrates, nitrofurantoin, nomifensine, norethindrone, norethindrone acetate, norgestrel, nylidrin, nystafin, orphenadrine, oxacillin, oxazepam, oxprenolol, oxymetazoline, oxyphenbutazone, pancrelipase, pantothenic acid, papaverine, para-aminosalicylic acid, paramethasone, paregoric, pemoline, penicillamine, penicillin, penicillin-v, pentobarbital, perphenazine, phenacetin, phenazopyridine, pheniramine, phenobarbital, phenolphthalein, phenprocoumon, phensuximide, phenylbutazone, phenylephrine, phenylpropanolamine, phenyl toloxamine, phenytoin, pilocarpine, pindolol, piper acetazine, piroxicam, poloxamer, polycarbophil calcium, polythiazide, potassium supplements, pruzepam, prazosin, prednisolone, prednisone, primidone, probenecid, probucol, procainamide, procarbazine, prochlorperazine, procyclidine, promazine, promethazine, propantheline, propranolol, pseudoephedrine, psoralens, syllium, pyridostigmine, pyrodoxine, pyrilamine, pyrvinium, quinestrol, quinethazone, uinidine, quinine, ranitidine, rauwolfia alkaloids, riboflavin, rifampin, ritodrine, alicylates, scopolamine, secobarbital, senna, sannosides a & b, simethicone, sodium bicarbonate, sodium phosphate, sodium fluoride, spironolactone, sucrulfate, sulfacytine, sulfamethoxazole, sulfasalazine, sulfinpyrazone, sulfisoxazole, sulindac, talbutal, tamazepam, terbutaline, terfenadine, terphinhydrate, teracyclines, thiabendazole, thiamine, thioridazine, thiothixene, thyroblobulin, thyroid, thyroxine, ticarcillin, timolol, tocainide, tolazamide, tolbutamide, tolmetin trozodone, tretinoin, triamcinolone, trianterene, triazolam, trichlormethiazide, tricyclic antidepressants, tridhexethyl, trifluoperazine, triflupromazine, trihexyphenidyl, trimeprazine, trimethobenzamine, trimethoprim, tripclennamine, triprolidine, valproic acid, verapamil, vitamin A, vitamin B₁₂, vitamin C, vitamin D, vitamin E, vitamin K, xanthine, and the like.

Diagnostic agents include gases; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include, but are not limited to, gadolinium chelates, as well as iron, magnesium, manganese, copper, and chromium. Non-limiting examples of materials useful for CAT and x-ray imaging include iodine-based materials.

Prophylactic agents include, for instance, vaccines, nutritional compounds, such as vitamins, antioxidants etc.

The releasable species may be delivered as a mixture in some cases, e.g., a mixture of pharmaceutically active releasable species. For instance, one or more releasable species may be present in a single article. Alternatively, a composition of articles may include multiple articles, each housing a single releasable species, but where more than one type of releasable species is present within the composition. For example, a local anesthetic may be delivered in combination with an anti-inflammatory agent such as a steroid in the same or separate articles. An antibiotic may be combined with an inhibitor of the enzyme commonly produced by bacteria to inactivate the antibiotic (e.g., penicillin and clavulanic acid).

As discussed, the article may be implanted into a subject, such as a human, according to one aspect of the invention. The article may be implanted in any suitable location within the subject, e.g., in an area where localized delivery of a drug or other releasable species from the article is needed, or in an area providing ready access to the bloodstream or to the brain, depending on the application. For instance, the article may be implanted subcutaneously, on or proximate a nerve or an organ, etc., or the article may be positioned on the surface of the skin in some cases. It should be understood, however, that the invention is not limited only to implant applications. For instance, the articles and pharmaceutical compositions containing articles may be administered to an individual via any route known in the art. These include, but are not limited to, oral, sublingual, nasal, intradermal, subcutaneous, intramuscular, rectal, vaginal, intravenous, intraarterial, and inhalational administration.

When administered to a site other than the intended site of therapy the articles of the invention, may be modified to include targeting agents to target the article to a particular cell, collection of cells, or tissue. A variety of targeting agents that direct pharmaceutical compositions to particular cells are known in the art (see, for example, Cotton, et al. Methods Enzym. 217:618, 1993; incorporated herein by reference). The targeting agents may be included throughout the particle or may be only on the surface. The targeting agent may be a protein, peptide, carbohydrate, glycoprotein, lipid, small molecule, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gpl 20 envelope protein of the human immunodeficiency virus (HIV), carbohydrates, receptor ligands, sialic acid, etc.

As used herein, a “subject,” means a human or non-human animal. Examples of subjects include, but are not limited to, a mammal such as a dog, a cat, a horse, a rabbit, a pig, a sheep, a rat, a mouse, a primate (e.g., a monkey, a chimpanzee, a baboon, an ape, a gorilla, etc.), or the like. The implantable article may thus contain one or more biocompatible materials. For instance, some or all of the magnetically susceptible material, the enclosure, and/or the heat-sensitive material may comprise biocompatible materials.

As used herein, “biocompatible” is given its ordinary meaning in the art. For instance, a biocompatible material is one that is suitable for implantation into a subject without adverse consequences, for example, without substantial acute or chronic inflammatory response and/or acute rejection of the fabric material by the immune system, for instance, via a T-cell response. It will be recognized, of course, that “biocompatibility” is a relative term, and some degree of inflammatory and/or immune response is to be expected even for materials that are highly compatible with living tissue. However, non-biocompatible materials are typically those materials that are highly inflammatory and/or are acutely rejected by the immune system, i.e., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In some cases, even if the material is not removed, the immune response by the subject is of such a degree that the material ceases to function; for example, the inflammatory and/or the immune response of the subject may create a fibrous “capsule” surrounding the material that effectively isolates it from the rest of the subject's body and thereby prevents proper release of the releasable species from the article; materials eliciting such a reaction would also not be considered as “biocompatible materials” as used herein.

The articles of the invention may be used to deliver a drug to the subject in an effective amount for treating disorders such as cancer and chronic disorders such as neurological disorders, diabetes, cardiovascular disorders, autoimmune disease and pain. An “effective amount,” for instance, is an amount necessary or sufficient to realize a desired biologic effect. An “effective amount for treating cancer,” for instance, could be that amount necessary to (i) prevent further cancer cell proliferation, survival and/or growth and/or (ii) arresting or slowing cancer cell proliferation, survival and/or growth with respect to cancer cell proliferation, survival and/or growth in the absence of the therapy. According to some embodiments of the invention, an effective amount is that amount of a compound of the invention alone or in combination with another medicament, which when combined or co-administered or administered alone, results in a therapeutic response to the disease, either in the prevention or the treatment of the disease. The biological effect may be the amelioration and or absolute elimination of symptoms resulting from the disease. In another embodiment, the biological effect is the complete abrogation of the disease, as evidenced, for example, by the absence of a symptom of the disease.

As used herein, the term “treating” and “treatment” refers to modulating certain tissues so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. One of skill in the art realizes that a treatment may improve the disease condition, but may not be a complete cure for the disease.

In some embodiments, the present invention provides a method of treating a cancer comprising administering to a subject in whom such treatment is desired a therapeutically effective amount of a composition of the invention. A composition of the invention may, for example, be used as a first, second, third or fourth line cancer treatment. In some embodiments, the invention provides methods for treating a cancer (including ameliorating a symptom thereof) in a subject refractory to one or more conventional therapies for such a cancer, said methods comprising administering to said subject a therapeutically effective amount of an article of the invention having one or more anti-cancer drugs therein. A cancer may be determined to be refractory to a therapy when at least some significant portion of the cancer cells are not killed or their cell division is not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased.

The invention also provides methods for treating cancer by administering an article of the invention in combination with any other anti-cancer treatment (e.g., radiation therapy, chemotherapy or surgery) to a patient. Cancers that can be treated by the methods encompassed by the invention include, but are not limited to, neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman, et al., 1985, Medicine, 2d Ed., J.B. Lippincott Co., Philadelphia).

Specific cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

The articles of the invention also can be administered to prevent progression to a neoplastic or malignant state. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79.). Hyperplasia is a form of controlled cell proliferation involving an increase in cell number in a tissue or organ, without significant alteration in structure or function. Endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

The prophylactic use of the articles of the invention is also indicated in some viral infections that may lead to cancer. For example, human papilloma virus can lead to cervical cancer (see, e.g., Hernandez-Avila et al., Archives of Medical Research (1997) 28: 265-271), Epstein-Barr virus (EBV) can lead to lymphoma (see, e.g., Herrmann et al., J. Pathol. (2003) 199(2):140-5), hepatitis B or C virus can lead to liver carcinoma (see, e.g., El-Serag, J. Clin. Gastroenterol. (2002) 35(5 Suppl 2): S72-8), human T cell leukemia virus (HTLV)-I can lead to T-cell leukemia (see e.g., Mortreux et al., Leukemia (2003) 17(1): 26-38), and human herpesvirus-8 infection can lead to Kaposi's sarcoma (see, e.g., Kadow et al., Curr. Opin. Investig. Drugs (2002) 3(11): 1574-9).

Examples of conventional anti-cancer agents which can be incorporated in the articles of the invention include methotrexate, trimetrexate, adriamycin, taxotere, doxorubicin, 5-flurouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, etc.

Another form of anti-cancer therapy involves administering an antibody specific for a cell surface antigen of, for example, a cancer cell. In one embodiment, the antibody incorporated in the article of the invention may be selected from the group consisting of Ributaxin, Herceptin, Rituximab, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA. Other antibodies include but are not limited to anti-CD20 antibodies, anti-CD40 antibodies, anti-CD19 antibodies, anti-CD22 antibodies, anti-HLA-DR antibodies, anti-CD80 antibodies, anti-CD86 antibodies, anti-CD54 antibodies, and anti-CD69 antibodies. These antibodies are available from commercial sources or may be synthesized de novo.

Examples of anti-cancer agents include, but are not limited to, DNA-interactive agents including, but not limited to, the alkylating agents (for example, nitrogen mustards, e.g. Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin interactive agents including, but not limited to, colcbicine, Vincristine and Vinblastine, both alkaloids and Paclitaxel and cytoxan; hormonal agents including, but note limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1 α, IL-1 β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, and Uteroglobins (U.S. Pat. No. 5,696,092); anti-angiogenics including, but not limited to, agents that inhibit VEGF (e.g., other neutralizing antibodies (Kim et al., 1992; Presta et al., 1997; Sioussat et al., 1993; Kondo et al., 1993; Asano et al., 1995, U.S. Pat. No. 5,520,914), soluble receptor constructs (Kendall and Thomas, 1993; Aiello et al., 1995; Lin et al., 1998; Millauer et al., 1996), tyrosine kinase inhibitors (Siemeister et al., 1998, U.S. Pat. Nos. 5,639,757, and 5,792,771), antisense strategies, RNA aptamers and ribozymes against VEGF or VEGF receptors (Saleh et al., 1996; Cheng et al., 1996; Ke et al., 1998; Parry et al., 1999); variants of VEGF with antagonistic properties as described in WO 98/16551; compounds of other chemical classes, e.g., steroids such as the angiostatic 4,9(11)-steroids and C21-oxygenated steroids, as described in U.S. Pat. No. 5,972,922; thalidomide and related compounds, precursors, analogs, metabolites and hydrolysis products, as described in U.S. Pat. Nos. 5,712,291 and 5,593,990;

Thrombospondin (TSP-1) and platelet factor 4 (PF4); interferons and metalloproteinsase inhibitors; tissue inhibitors of metalloproteinases (TIMPs); anti-Invasive Factor, retinoic acids and paclitaxel (U.S. Pat. No. 5,716,981); AGM-1470 (Ingber et al., 1990); shark cartilage extract (U.S. Pat. No. 5,618,925); anionic polyamide or polyurea oligomers (U.S. Pat. No. 5,593,664); oxindole derivatives (U.S. Pat. No. 5,576,330); estradiol derivatives (U.S. Pat. No. 5,504,074); thiazolopyrimidine derivatives (U.S. Pat. No. 5,599,813); and LM609 (U.S. Pat. No. 5,753,230); apoptosis-inducing agents including, but not limited to, bcr-abl, bcl-2 (distinct from bcl-1, cyclin D1; GenBank accession numbers M14745, X06487; U.S. Pat. Nos. 5,650,491; and 5,539,094) and family members including Bcl-xl, Mcl-1, Bak, A1, A20, and antisense nucleotide sequences (U.S. Pat. Nos. 5,650,491; 5,539,094; and 5,583,034); Immunotoxins and coaguligands, tumor vaccines, and antibodies.

Cancer therapies and their dosages, and recommended usage are known in the art and have been described in such literature as the Physician's Desk Reference (56^{th} ed., 2002), which is incorporated by reference.

The term “neurological disorder” as used in this invention includes neurological diseases, neurodegenerative diseases, and neuropsychiatric disorders. A neurological disorder is a condition having as a component a central or peripheral nervous system malfunction. Neurological disorders may cause a disturbance in the structure or function of the nervous system resulting from developmental abnormalities, disease, genetic defects, injury or toxin. These disorders may affect the central nervous system (e.g., the brain, brainstem and cerebellum), the peripheral nervous system (e.g., the cranial nerves, spinal nerves, and sympathetic and parasympathetic nervous systems) and/or the autonomic nervous system (e.g., the part of the nervous system that regulates involuntary action and that is divided into the sympathetic and parasympathetic nervous systems).

As used herein the term “neurodegenerative disease” implies any disorder that might be reversed, deterred, managed, treated, improved, or eliminated with agents that stimulate the generation of new neurons. Examples of neurodegenerative disorders include: (i) chronic neurodegenerative diseases such as familial and sporadic amyotrophic lateral sclerosis (FALS and ALS, respectively), familial and sporadic Parkinson's disease, Huntington's disease, familial and sporadic Alzheimer's disease, multiple sclerosis, olivopontocerebellar atrophy, multiple system atrophy, progressive supranuclear palsy, diffuse Lewy body disease, corticodentatonigral degeneration, progressive familial myoclonic epilepsy, strionigral degeneration, torsion dystonia, familial tremor, Down's Syndrome, Gilles de la Tourette syndrome, Hallervorden-Spatz disease, diabetic peripheral neuropathy, dementia pugilistica, AIDS Dementia, age related dementia, age associated memory impairment, and amyloidosis-related neurodegenerative diseases such as those caused by the prion protein (PrP) which is associated with transmissible spongiform encephalopathy (Creutzfeldt-Jakob disease, Gerstmann-Straussler-Scheinker syndrome, scrapic, and kuru), and those caused by excess cystatin C accumulation (hereditary cystatin C angiopathy); and (ii) acute neurodegenerative disorders such as traumatic brain injury (e.g., surgery-related brain injury), cerebral edema, peripheral nerve damage, spinal cord injury, Leigh's disease, Guillain-Barre syndrome, lysosomal storage disorders such as lipofuscinosis, Alper's disease, vertigo as result of CNS degeneration; pathologies arising with chronic alcohol or drug abuse including, for example, the degeneration of neurons in locus coeruleus and cerebellum; pathologies arising with aging including degeneration of cerebellar neurons and cortical neurons leading to cognitive and motor impairments; and pathologies arising with chronic amphetamine abuse including degeneration of basal ganglia neurons leading to motor impairments; pathological changes resulting from focal trauma such as stroke, focal ischemia, vascular insufficiency, hypoxic-ischemic encephalopathy, hyperglycemia, hypoglycemia or direct trauma; pathologies arising as a negative side-effect of therapeutic drugs and treatments (e.g., degeneration of cingulate and entorhinal cortex neurons in response to anticonvulsant doses of antagonists of the NMDA class of glutamate receptor). and Wernicke-Korsakoff's related dementia. Neurodegenerative diseases affecting sensory neurons include Friedreich's ataxia, diabetes, peripheral neuropathy, and retinal neuronal degeneration. Other neurodegenerative diseases include nerve injury or trauma associated with spinal cord injury. Neurodegenerative diseases of limbic and cortical systems include cerebral amyloidosis, Pick's atrophy, and Retts syndrome. The foregoing examples are not meant to be comprehensive but serve merely as an illustration of the term “neurodegenerative disorder.”

Parkinson's disease is a disturbance of voluntary movement in which muscles become stiff and sluggish. Symptoms of the disease include difficult and uncontrollable rhythmic twitching of groups of muscles that produces shaking or tremors. Currently, the disease is caused by degeneration of pre-synaptic dopaminergic neurons in the brain and specifically in the brain stem. As a result of the degeneration, an inadequate release of the chemical transmitter dopamine occurs during neuronal activity.

Currently, Parkinson's disease is treated with several different compounds and combinations. Levodopa (L-dopa), which is converted into dopamine in the brain, is often given to restore muscle control. Perindopril, an ACE inhibitor that crosses the blood-brain barrier, is used to improve patients' motor responses to L-dopa. Carbidopa is administered with L-dopa in order to delay the conversion of L-dopa to dopamine until it reaches the brain, and it also lessens the side effects of L-dopa. Other drugs used in Parkinson's disease treatment include dopamine mimickers Mirapex (pramipexole dihydrochloride) and Requip (ropinirole hydrochloride), and Tasmar (tolcapone), a COMT inhibitor that blocks a key enzyme responsible for breaking down levodopa before it reaches the brain.

One group of neuropsychiatric disorders includes disorders of thinking and cognition, such as schizophrenia and delirium. A second group of neuropsychiatric disorders includes disorders of mood, such as affective disorders and anxiety. A third group of neuropsychiatric disorders includes disorders of social behavior, such as character defects and personality disorders. And a fourth group of neuropsychiatric disorders includes disorders of learning, memory, and intelligence, such as mental retardation and dementia. Accordingly, neuropsychiatric disorders encompass schizophrenia, delirium, attention deficit disorder (ADD), schizoaffective disorder Alzheimer's disease, depression, mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder and disinhibition.

Examples of antipsychotic drugs that may be used to treat schizophrenic patients include phenothizines, such as chlorpromazine and trifluopromazine; thioxanthenes, such as chlorprothixene; fluphenazine; butyropenones, such as haloperidol; loxapine; mesoridazine; molindone; quetiapine; thiothixene; trifluoperazine; perphenazine; thioridazine; risperidone; dibenzodiazepines, such as clozapine; and olanzapine. Benzodiazepines, which enhance the inhibitory effects of the gamma aminobutyric acid (GABA) type A receptor, are frequently used to treat anxiety. Buspirone is another effective anxiety treatment.

According to an embodiment of the invention, the methods described herein are useful in treating autoimmune disease in a subject by administering an article of the invention to the subject. Thus, the methods are useful for such autoimmune diseases as multiple sclerosis, systemic lupus erythematosus, type 1 diabetes, viral endocarditis, viral encephalitis, rheumatoid arthritis, Graves' disease, autoimmune thyroiditis, autoimmune myositis, and discoid lupus erythematosus.

“Autoimmune Disease” refers to those diseases which are commonly associated with the nonanaphylactic hypersensitivity reactions (Type II, Type III and/or Type IV hypersensitivity reactions) that generally result as a consequence of the subject's own humoral and/or cell-mediated immune response to one or more immunogenic substances of endogenous and/or exogenous origin. Such autoimmune diseases are distinguished from diseases associated with the anaphylactic (Type I or IgE-mediated) hypersensitivity reactions.

The articles of the invention are also useful in the treatment of diabetes. Diabetes is a chronic metabolic disorder which includes a severe form of childhood diabetes (also called juvenile, Type I or insulin-dependent diabetes). Type II Diabetes (DM II) is generally found in adults. Patients with diabetes of all types have considerable morbidity and mortality from microvascular (retinopathy, neuropathy, nephropathy) and macrovascular (heart attacks, stroke, peripheral vascular disease) pathology. Non-insulin dependent diabetes mellitus develops especially in subjects with insulin resistance and a cluster of cardiovascular risk factors such as obesity, hypertension and dyslipidemia, a syndrome which first recently has been recognized and is named “the metabolic syndrome.”

Antidiabetic agents, include insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; protein tyrosine phosphatase-1 B (PTP-1 B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose cotransporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; and DPPIV (dipeptidyl peptidase IV) inhibitors such as LAF237;b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;c) anti-obesity agents such as orlistat; and) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as ditekiren, zankiren, terlakiren, aliskiren, RO 66-1132 and RO-66-1168; beta-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors.

Cardiovascular disorders, treatable using the articles of the invention, include but are not limited to disorders of the heart and the vascular system like congestive heart failure, myocardial infarction, ischemic diseases of the heart, all kinds of atrial and ventricular arrhythmias, hypertensive vascular diseases, peripheral vascular diseases, and atherosclerosis. Heart failure is a pathophysiological state in which an abnormality of cardiac function is responsible for the failure of the heart to pump blood at a rate commensurate with the requirement of the metabolizing tissue. It includes all forms of pumping failures such as high-output and low-output, acute and chronic, right-sided or left-sided, systolic or diastolic, independent of the underlying cause. Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included as well as the acute treatment of MI and the prevention of complications. Ischemic disease is a condition in which the coronary flow is restricted resulting in a perfusion which is inadequate to meet the myocardial requirement for oxygen, such as stable angina, unstable angina and asymptomatic ischemia. Arrhythmias include atrial and ventricular tachyarrhythmias, atrial tachycardia, atrial flutter, atrial fibrillation, atrio-ventricular reentrant tachycardia, preexitation syndrome, ventricular tachycardia, ventricular flutter, ventricular fibrillation, as well as bradycardic forms of arrhythmias. Hypertensive vascular diseases include primary as well as all kinds of secondary arterial hypertension, renal, endocrine, neurogenic, others. Peripheral vascular diseases are vascular diseases in which arterial and/or venous flow is reduced resulting in an imbalance between blood supply and tissue oxygen demand and include chronic peripheral arterial occlusive disease (PAOD), acute arterial thrombosis and embolism, inflammatory vascular disorders, Raynaud's phenomenon and venous disorders. Atherosclerosis is a cardiovascular disease in which the vessel wall is remodeled, compromising the lumen of the vessel.

In one embodiment, articles containing an anesthetic (e.g., bupivacaine, levobupivacaine, lidocaine, mepivacaine, ropivacaine, tetracaine, prilocaine, ropivacaine, articaine, trimecaine and their salts and prodrugs) are administered in the vicinity of a nerve to provide a nerve block. Nerve blocks provide a method of anesthetizing large areas of the body without the risks associated with general anesthesia. Any nerve may be anesthetized in this manner. The articles containing the releasable species are deposited as close to the nerve as possible without injecting directly into the nerve. Particularly preferred nerves include the sciatic nerve, the femoral nerve, inferior alveolar nerve, nerves of the brachial plexus, intercostal nerves, nerves of the cervical plexus, median nerve, ulnar nerve, and sensory cranial nerves. In an embodiment, epinephrine or another vasoactive agent may be administered along with the local anesthetic to prolong the block. The epinephrine or other agent (e.g., other vasoactive agents, steroidal compounds, non-steroidal anti-inflammatory compounds) may be encapsulated in the articles containing the local anesthetic, encapsulated in articles by itself, or unencapsulated. Additionally a pharmaceutically effective glucocorticosteroid is administered locally or systemically, to a patient, before any local anesthetic is administered to the patient. In this aspect, the glucocorticosteroid dose will then potentiate, e.g., prolong the duration or increase the degree of anesthesia of a later-administered local anesthetic. One of ordinary skill in this art would be able to determine the choice of anesthetic as well as the amount and concentration of anesthetic based on the nerves and types of nerve fibers to be blocked, the duration of anesthesia required, and the size and health of the patient (Hardman & Limbird, Eds., Goodman & Gilman's The Pharmacological Basis of Therapeutics Ninth Edition, Chapter 15, pp. 331-347, 1996; incorporated herein by reference). As used herein, the term “anesthetic agent” means any drug or mixture of drugs that provides numbness and/or analgesia. Examples of anesthetic agents which can be used include bupivacaine, levobupivacaine, lidocaine, mepivacaine, ropivacaine, tetracaine, prilocaine, ropivacaine, articaine, trimecaine and their salts and prodrugs, and mixtures thereof and any other art-known pharmaceutically acceptable anesthetic. The anesthetic can be in the form of a salt, for example, the hydrochloride, bromide, acetate, citrate, carbonate or sulfate. More preferably, the anesthetic agent is in the form of a free base.

The dose of anesthetic includes within the article of the invention will depend on the particular type of anesthetic as well as the objectives of the treatment. For example, when the drug included in the articles of the present invention is bupivacaine, the formulation may include, e.g., from about 0.5 to about 2 mg/kg body weight. Since the formulations of the present invention are controlled release, it is contemplated that formulations may include much more than usual immediate release doses, e.g., as much as 450 mg/kg anesthetic or more. The effective dose of anesthetic sufficient to provide equivalent potency (i.e., equally effective doses), can range from about 1 to about 50 mg injected or inserted at each site where the release of anesthetic agent is desired.

The compositions of the invention can generally be used in any art known procedures for anesthetizing a patient. For example, they may be used for infiltration anesthesia, wherein a formulation suitable for injection is injected directly into the tissue requiring anesthesia. For example, an effective amount of the formulation in injectable form is infiltrated into a tissue area that is to be incised or otherwise requires anesthesia. In addition, the anesthetic formulations and methods according to the invention can be used for field block anesthesia, by injecting an effective amount of the formulation in injectable form in such a manner as to interrupt nerve transmission proximal to the site to be anesthetized. For instance, subcutaneous infiltration of the proximal portion of the volar surface of the forearm results in an extensive area of cutaneous anesthesia that starts 2 to 3 cm distal to the site of injection. Simply by way of example, the same effect can be achieved for the scalp, anterior abdominal wall and in the lower extremities.

Further, for even more efficient results, the local anesthetic formulations and methods according to the invention can be used for nerve block anesthesia. For example, an effective amount of the formulation in injectable form is injected into or adjacent to individual peripheral nerves or nerve plexuses. Injection of an effective amount of an anesthetic formulation according to the invention into mixed peripheral nerves and nerve plexuses can also desirably anesthetize somatic motor nerves, when required. The formulations and methods according to the invention can also be used for intravenous regional anesthesia by injecting a pharmacologically effective amount of microspheres in injectable form into a vein of an extremity that is subjected to a tourniquet to occlude arterial flow. Further still, spinal and epidural anesthesia using formulations, e.g., injectable compositions will be appreciated by the artisan to be within the scope contemplated by the present invention.

The articles may be used alone or combined with other pharmaceutical excipients, such as a pharmaceutically acceptable excipient or carrier, to form a pharmaceutical composition. As would be appreciated by one of skill in this art, the excipients may be chosen based on the route of administration, the releasable species being delivered, the time course of delivery of the releasable species, etc. As used herein, the term “pharmaceutically acceptable carrier” means a non-toxic, inert solid, semi-solid or liquid filler, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Implanted articles, may be implanted directly or formulated and then implanted. If an article is injected, the articles may also be formulated or injected alone. Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In a particularly preferred embodiment, the articles are suspended in a carrier fluid comprising 1% (w/v) sodium carboxymethyl cellulose and 0.1% (v/v) Tween 80.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

If the articles are delivered to a subject by alternative routes, they may be prepared in formulations suitable or oral, rectal, vaginal, nasal, subcutaneous, or pulmonary delivery. Liquid dosage forms for oral administration include pharmaceutically acceptable emulsions, microemulsions, solutions, suspensions, syrups, and elixirs. In addition to the active ingredients (i.e., articles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories which can be prepared by mixing the articles with suitable non-irritating excipients or carriers such as cocoa butter, polyethylene glycol, or a suppository wax which are solid at ambient temperature but liquid at body temperature and therefore melt in the rectum or vaginal cavity and release the articles.

Solid compositions of a similar type may also be employed as fillers in soft and hard-filled gelatin capsules using such excipients as lactose or milk sugar as well as high molecular weight polyethylene glycols and the like.

Incorporated herein by reference in their entireties are U.S. Provisional Patent Application Ser. No. 61/166,504, filed on Apr. 3, 2009, entitled “Radiative Heating for Drug Delivery and Other Applications,” by Hoare, et al.; U.S. Provisional Patent Application Ser. No. 61/166,428, filed on Apr. 3, 2009, entitled “Heating of Polymers and Other Materials using Radiation for Drug Delivery and Other Applications,” by Hoare, et al.; U.S. Provisional Patent Application Ser. No. 61/166,526, filed Apr. 3, 2009, entitled “Magnetic Heating for Drug Delivery and Other Applications,” by Hoare, et al.; and U.S. Provisional Patent Application Ser. No. 61/083,458, filed Jul. 24, 2008, entitled “Externally-Triggered Thermosensitive Membranes,” by Hoare, et al. Also incorporated herein by reference in their entireties are a PCT application filed on even date herewith, entitled “Radiative Heating for Drug Delivery and Other Applications,” by Hoare, et al.; and a PCT application filed on even date herewith, entitled “Heating of Polymers and Other Materials using Radiation for Drug Delivery and Other Applications,” by Hoare, et al.

The following examples are intended to illustrate certain embodiments of the present invention, but do not exemplify the full scope of the invention.

### EXAMPLE 1

This example describes the design and fabrication of membranes and drug delivery devices made thereof, which can be externally triggered to release a specific amount of a given drug at a desired site inside the body via the application of electromagnetic radiation. The application of an external heat source (including, but not limited to, dipole heating of a ferrofluid in an oscillating magnetic field, or direct heating by a heating pad or bath) can be used to open the pores of a membrane in which the pores are filled with a network of thermosensitive gel particles, increasing the flux of a drug contained within the device reservoir. Such a device can allow for external, “on/off” temporal control of drug delivery in vivo with drug release in the “on” state exhibiting a constant, zero-order (or other) kinetics profile. This membrane and the associated device, in some embodiments, represent an electronics-free, implantable device, which can facilitate effective, localized, rapid, non-invasive, repeatable, and “on-demand” drug release over long periods of time without requiring injections or negatively affecting other regions of the body or surrounding tissues.

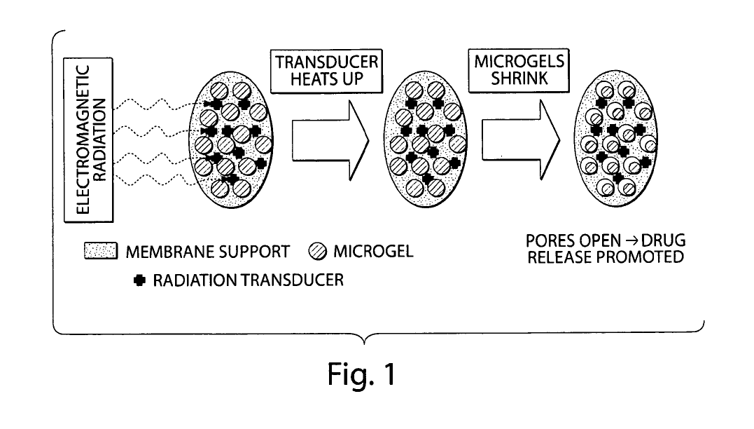

The device in this particular example comprised a composite membrane comprising a polymer backbone, a thermosensitive microgel, and a heat transducer (for example, a magnetic ferrofluid). The membrane was cast such that the pores of the membrane were at least partially filled with the thermosensitive microgels, which, for example, had diameters of about 800 nm in the swollen state (e.g., less than 37° C.) and diameters of about 250-300 nm in the collapsed state (e.g., greater than 42° C.). These temperatures can be changed as desired by copolymerization of different comonomers into the microgel network; more hydrophilic or stiffer monomers may result in higher transition temperatures, while more hydrophobic or flexible monomers may reduce the transition temperature. The magnetic or metallic particles were incorporated throughout the bulk of the membrane such that they do not interfere with the thermal swelling of the microgels. Without wishing to be bound by any theory, the resulting polymer membrane was believed to work as follows. (1) The inorganic additives in the membrane (e.g., ferrofluid) emitted heat in the presence of an applied magnetic or electromagnetic field. An oscillating magnetic field created heat via energy level transitions due to dipole switching in the ferromagnetic material, whereas microwaves created heat via resistive heating of the conductive gold nanoparticles. (2) Heat was transferred from the inorganic additives to the microgels adjacent to them in the membrane design, causing the thermosensitive microgel to undergo a deswelling volume phase transition and reduce its volume. (3) The reduced volume of the microgel increased the free volume within the fixed-size pores of the polymer membrane (defined by the polymer backbone), increasing the rate of drug diffusion through the membrane. When the electromagnetic radiation or the oscillating magnetic field was removed, the device cooled by thermal conduction to the cooler environment, causing the thermosensitive microgel to swell back to its original volume and fill the pores of the membrane. Consequently, the free volume in the membrane was decreased and the drug diffusion rate decreased. This membrane design and activation scheme, according to one embodiment, is summarized in FIG. 1.

To apply this technology in drug delivery applications, a reservoir drug delivery device based on this membrane was designed that can regulate the release of an active agent (e.g., a drug) over a period of several days, several weeks, or several months. A device was constructed comprising a biocompatible silicone tube with the polymer membrane bonded to the ends, into which a composition, for example a drug solution or a supersaturated drug slurry, may be readily incorporated. Other devices embraced by the description herein could be composed of the membrane because of the flexible nature and mechanical strength of the membranes in the hydrated state. The device could be refilled as desired to provide longer term drug release. Furthermore, since the polymer membrane is a tough and flexible film, it can be cut into any shape while retaining its physical properties.

To make thermosensitive microgels, 0.9 g N-isopropylacrylamide (NIPAM), 0.5 g N-isopropylmethacrylamide (NIPMAM), 0.08 g N,N-methylenebisacrylamide (MBA), and 150 mL water were dissolved in a 500 mL round-bottom flask equipped with a magnetic stirrer. The mixture was placed under nitrogen for 30 minutes and heated to 70° C. under 200 RPM mixing. Ammonium persulfate (0.1 g) was then dissolved in 5 mL of water and injected into the flask to initiate the reaction. The reaction proceeded overnight, at which point the microgel suspension was cooled and dialyzed using a 500,000 Da MWCO membrane against distilled water to remove unreacted monomers and linear polymer by-products. The purified microgel was then lyophilized and reconstituted at desired concentrations in ethanol. Microgels with a physiological transition temperature were also prepared by copolymerizing N,N-dimethylacrylamide (DMA) with NIPAM as well as NIPAM, acrylamide, and either NIPMAM or DMA.

To prepare the ferrofluid, 3.04 g of FeCl₃ and 1.98 g of FeCl₂ were dissolved in 12.5 mL of distilled water and mixed at 500 RPM. Ammonium hydroxide (6.5 mL) was then added dropwise over a 10 minute period. After 10 additional minutes of mixing, 1 g of 8000 Da molecular weight PEO was added in 10 mL of water and the mixture was heated to 70° C. for 2 hours to promote adsorption of the PEO to the ferrofluid surface. The heat was then removed and the ferrofluid was mixed overnight. The ferrofluid was purified via magnetic separation (5 cycles) and concentrated to about 10 wt %.

For the optimal membrane formulation, 1.3 g of a 10 wt % ethylcellulose solution in ethanol was mixed with 0.87 mL of a 60 mg/mL microgel suspension in a petri dish. The ratio of ethylcellulose to microgel could be altered to control the permeability of the membrane; the higher the microgel:ethylcellulose ratio, the higher the flux. For ferrofluid-loaded membranes, 0.5 mL of the concentrated ferrofluid suspension was mixed with 0.5 mL of ethanol in an eppendorf tube and subsequently added dropwise to the ethylcellulose-microgel mixture and mixed until homogeneous. The membranes were then dried inside an unsealed Tupperware container to facilitate slow evaporation of the ethanol. The dried membranes were then lifted out of the petri dishes with a spatula and punched to the desired dimensions. A range of different formulations containing up to 50% ferrofluid or up to 40% microgel were fabricated and tested.

Glass flow cells were used to test the flux properties of the membranes. A membrane was compressed between two cells of equal volume (3.4 mL) using rubber washers and a clamp to ensure a tight seal. The flow cells were then filled with phosphate buffered saline (PBS), equipped with magnetic stirrers, and submerged in a bath at a target temperature. A total of 50 microliters of a 100 mg/mL sodium fluorescein solution was then typically added to one side of the flow chamber. After pre-determined time intervals, samples were taken from the receiving chamber of the flow cell to track the flux as a function of time. The flux is measured by UVNIS absorbance at 490 nm (fluorescein) or 262 nm (bupivacaine). Experiments were also performed using dextran-FITC (4000 Da molecular weight) and bupivacaine (10 mg/mL total solution in saline) as the test chemicals.

The devices were constructed as follows. Two 1 cm diameter disks were punched out of a membrane sheet produced as described above. A 1 cm length of ⅜ inch OD (¼ inch ID) silicone tubing (currently used for catheters) was then cut and a membrane disk was glued to one side of the tube using a LockTite low viscosity quick drying adhesive. (1 inch =2.54 cm) After 30 minutes of drying (under light pressure), the membrane-backed tube was then filled with drug or indicator solution. Sodium fluorescein (100 mg/mL in saline) and bupivacaine (10 mg/mL in saline as well as bupivacaine powder in a saturated saline solution with solid chunks of drug also added to give a larger drug reservoir for release) were tested. The top membrane was then attached using glue following the same technique as for the bottom membrane and set for 30 minutes under light pressure.

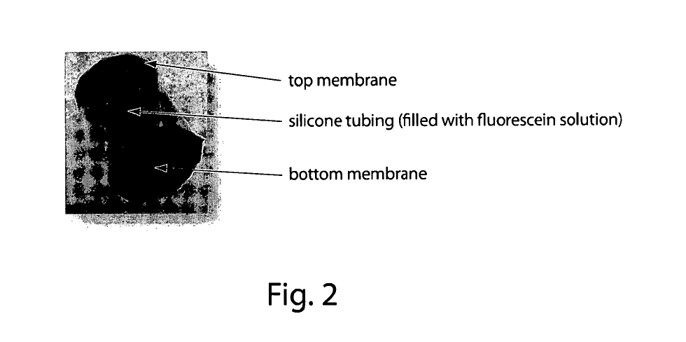

FIG. 2 shows a typical device. For flux testing, the devices were submerged in 5 mL of PBS at a specific test temperature, with samples taken at predetermined intervals to track the release kinetics.

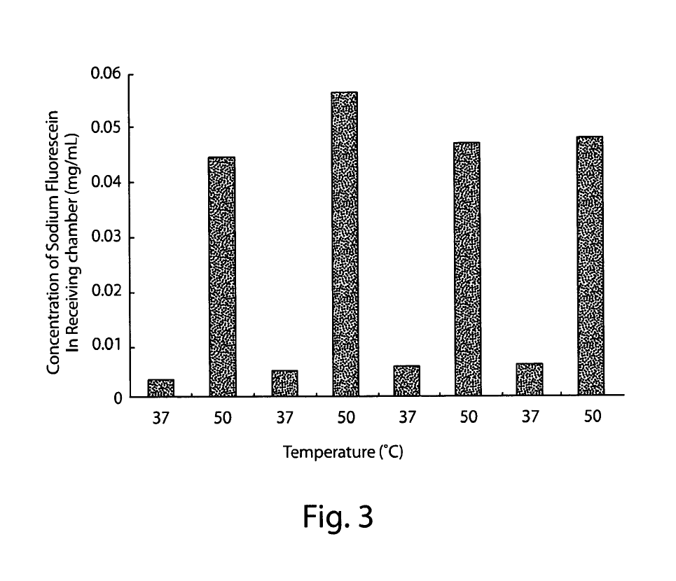

FIG. 3 shows the flux results for sodium fluorescein across a membrane containing about 31% ferrofluid and 25% microgel (dry mass) by weight, as measured via a flow cell experiment. The low temperature (“off”) state is body temperature (37° C.) while the high (“on”) temperature used for the test is 45° C., the highest temperature typically used as a control in hyperthermia studies. Similar flux results are achieved using 42° C.-45° C. as the high, “on” temperature. Time points between data points are 24 hours. As shown, the membrane had an approximately 20:1 flux differential between the “on” and “off” states.

It should be noted that the ferrofluid loading in this membrane (about 30%) does not represent an upper limit to the ferrofluid loading capacity of the membrane. The ferrofluid was primarily entrapped in the ethylcellulose around the microgel pores as opposed to in the microgel itself (as is typically the case for hydrogel thermal triggering), making the membrane more stable as well as more predictable in terms of heating since the phase transition temperature of the microgels can be strongly affected by the presence of a dopant (such as embedded ferrofluid).

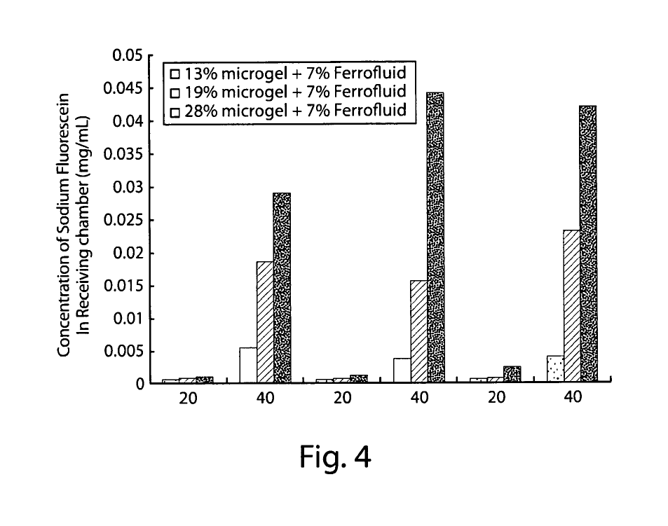

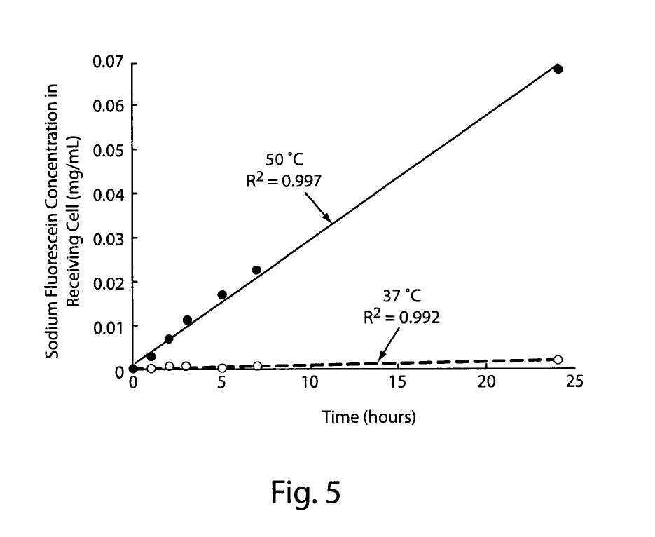

A range of microgels were tested with variations in properties such as gel loading (i.e. percent microgel per total mass), gel composition, active temperature range, and ferrofluid loading. The degree of drug flux can be controlled by changing the amount of microgel inside the membrane, as shown in FIG. 4. The microgel used to generate the data shown in FIG. 4 has a lower transition temperature (32° C.), but similar trends were observed with all microgels. Higher rates of flux could be achieved by adding more microgels. At very high microgel loadings the flux ratio between the low and high temperature decreased even as the absolute flux at the “on” state increased (i.e. the microgels are more “leaky” at low temperature), which may suggest an upper limit of ˜40 wt % microgel in the membranes. Drug release from the reservoirs was also shown to be linear both in the “off” state and in the “on” state, as illustrated in FIG. 5. This shows that it was possible to achieve zero-order release kinetics from the device over the course of at least one day.

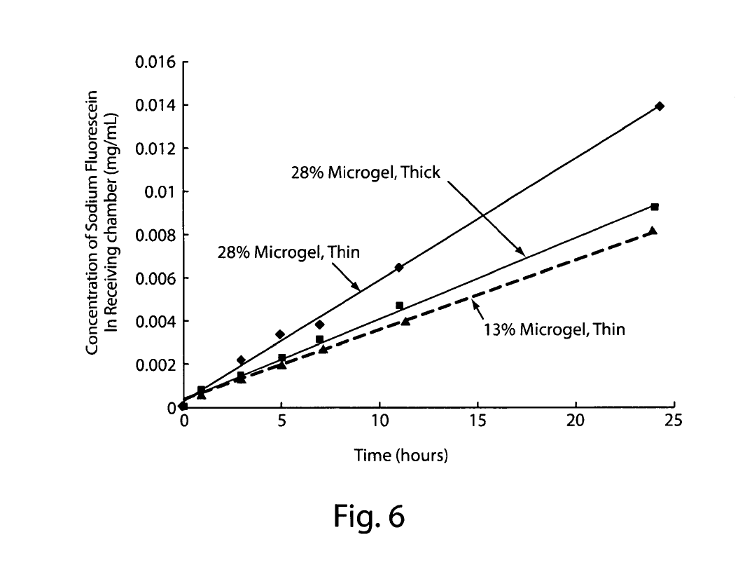

The release rate at a given temperature can also be controlled by changing the amount of microgel in the membranes and/or the thickness of the membranes, as shown in FIG. 6. The “thin” membrane was prepared at the default membrane thickness (0.13 g ethyl cellulose, about 0.15 mm thick) while the “thick” membrane was prepared with 0.23 g ethyl cellulose (about 0.25 mm thick). Membranes can be cast with any thickness desired to control flux and mechanical properties. As shown, thicker membranes released fluorescein slower than thinner membranes, as expected given the increased tortuosity of the pore structure a given molecule would have to diffuse through the membrane. Thus, flux control could be achieved by changing both the amount of microgel in the membrane and the thickness of the membrane.

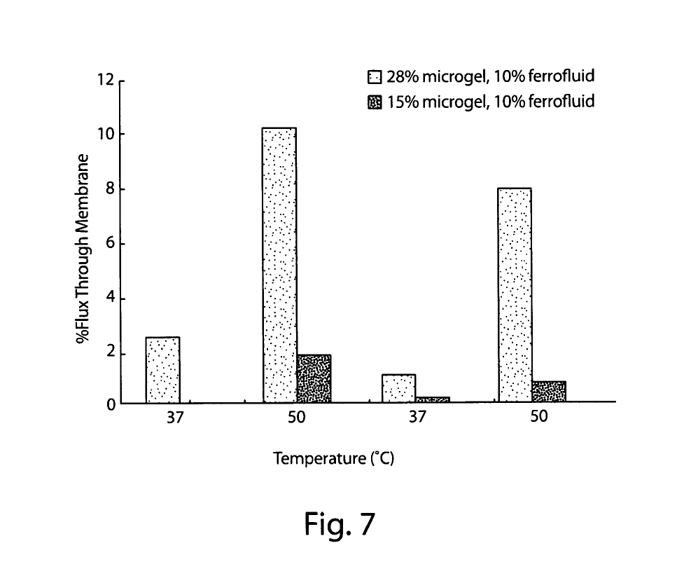

Although all the above results are shown for sodium fluorescein (MW=376 g/mol), larger molecules can also be released using this membrane technology. FIG. 7 shows flux results for dextran-FITC of molecular weight 4000 g/mol. The graph is expressed in terms of percent flux of the total amount of dextran-FITC added to the source chamber of the flow cell after 24 hours. As with the low molecular weight drug, more flux was observed through membranes with higher microgel loadings and increased flux was noted at high temperatures (in this case, the high temperature is 50° C. and the low temperature is 41° C., given the higher transition temperature of the particular microgel used for this experiment). Hence, the membranes are useful not only for delivering small molecule drugs but also macromolecular drugs such as insulin (molecular weight 5.8 kDa).

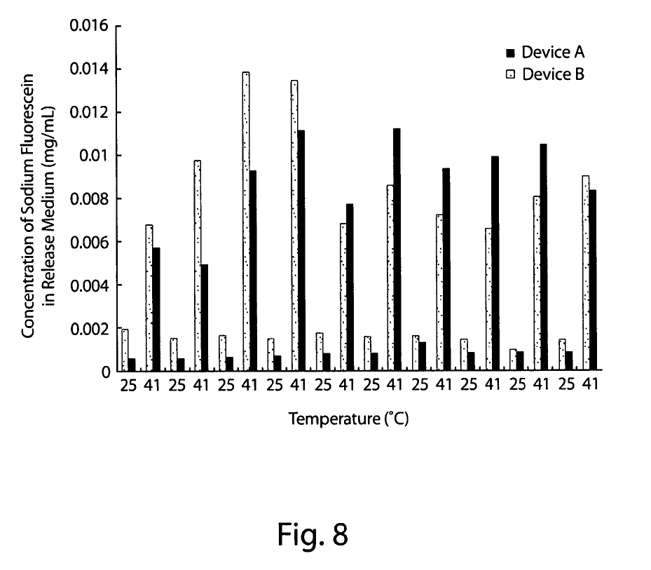

The flux results for a prototype device loaded with sodium fluorescein are shown in FIG. 8. A total of 10 thermal cycles with cycle times of 24 hours are shown. The two sets of bars represent flux results from two different devices fabricated with the same microgel. The low temperature membrane was used for this proof-of-concept experiment, although similar results were shown for the physiological temperature cycling devices over three thermal cycles.

The cycle-to-cycle reproducibility and the flux similarity between the two duplicate devices suggested that the devices exhibited reproducible behavior over a large number of thermal cycles. It should be noted that the experiment was arbitrarily ended after 10 total cycles even though the devices still contained a significant amount of sodium fluorescein and were not leaky, suggesting that more cycles were likely possible.

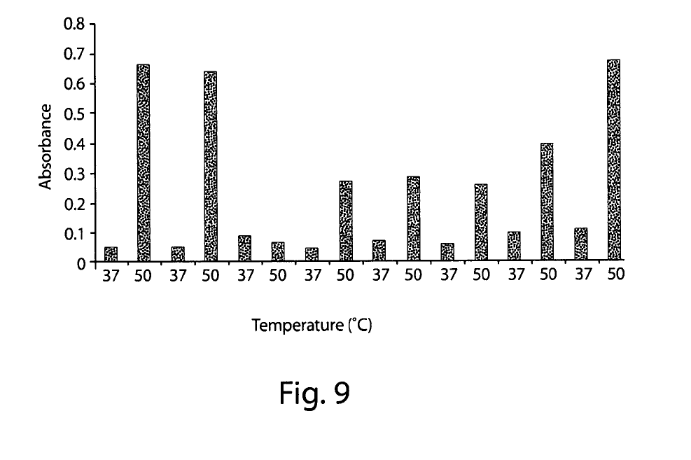

Miniaturization of the device to make it more amenable to implantation in smaller sites within the body (for example, at the sciatic nerve for the delivery of local anesthesia) was also investigated. FIG. 9 shows flux results (sodium fluorescein payload) from devices fabricated using a 3/32 inch ID-5/32 inch OD silicone tubing (also 1 cm length) as the device casing, using a physiologically triggerable membrane as the gating mechanism.

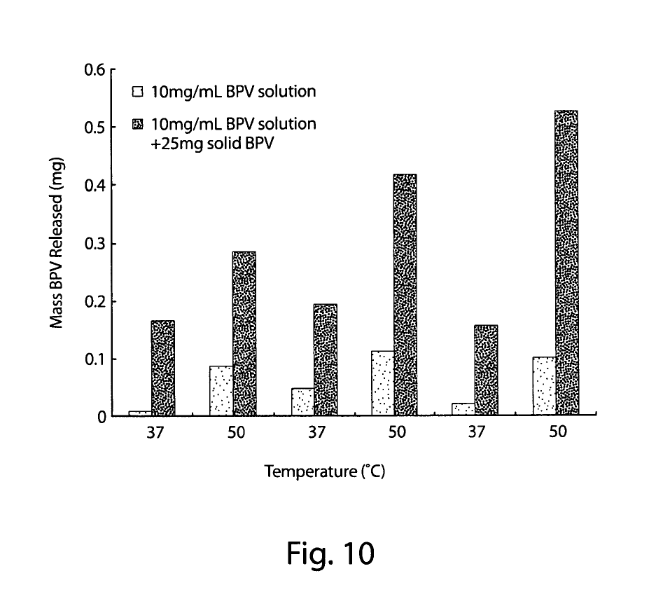

Differential release of the anesthetic bupivacaine into PBS (again using the physiologically-triggerable membrane) was also achieved using one of these devices, as shown in FIG. 10. In this experiment, a saturated bupivacaine solution in saline was loaded into the device together with an additional 25 mg of dry bupivacaine powder. Over time, the two-way nature of the membrane flux permitted inflow of water to dilute the high concentration of bupivacaine inside the device and dissolve the crystals, thereby permitting higher levels of bupivacaine release over a longer period of time than would be possible using only solution loading. For demonstration purposes, the flux could be tuned according to the microgel content of the membrane such that the release at 37° C. was below the minimal effective dose while release at 50° C. is above the minimal effective dose. Furthermore, although the same membranes were used for both bupivacaine and sodium fluorescein release, bupivacaine was significantly smaller than sodium fluorescein (MW=288 g/mol) and is cationic instead of anionic.

### EXAMPLE 2

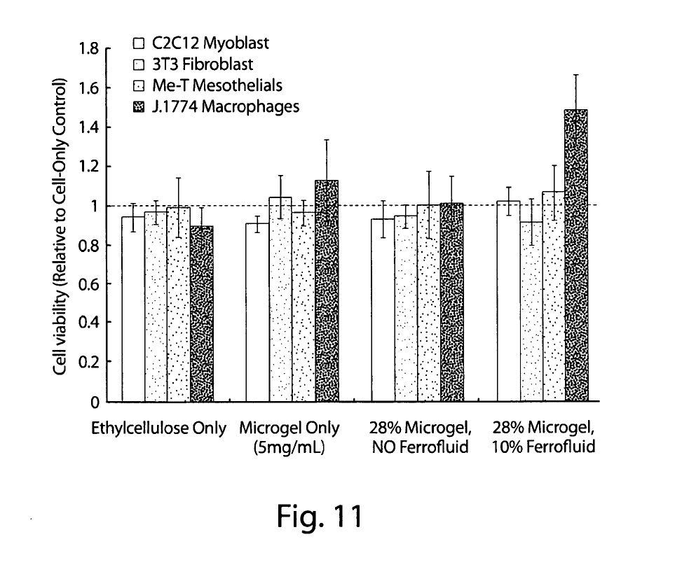

This example demonstrates the inertness of the membranes in cell and animal implant experiments. FIG. 11 shows results from an MTT metabolic activity assay on a range of different cell types likely to be present at or near the site of a subcutaneous or intramuscular implant (muscle cells, fibroblasts, macrophages, and mesothelial cells for peritoneal applications). The y-axis represents the ratio between the MTT signal from a well from cells exposed to the membrane compositions listed on the x-axis and the signal from cells (grown on the same plate), which were not exposed to any materials. Data was collected after 1 day of material exposure. In each case, the relative absorbance (normalized to cells grown in the absence of the membrane material) was approximately equal to one for all tested membranes with myotubes (differentiated muscle cells), fibroblasts, and mesothelial cells, suggesting that cell viability was not significantly impacted by the presence of the membrane. The slight increase in activity from the macrophage assay may indicate some macrophage activation by the presence of the foreign material, but was not large enough to be conclusive. Thus, there was no apparent in vitro problem with biocompatibility of the membrane components or the membrane itself

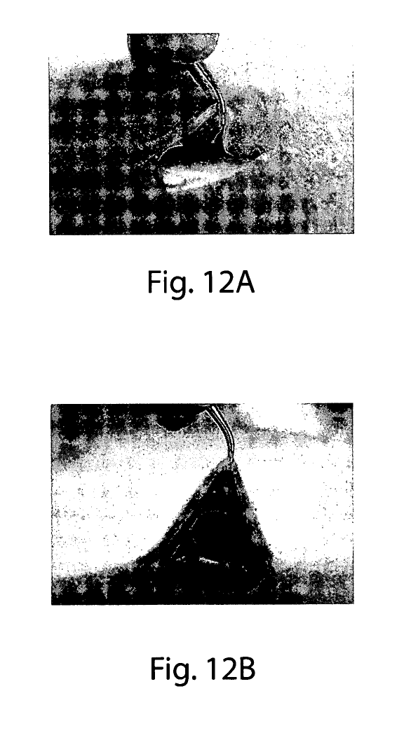

Membranes impregnated with ferrofluids were implanted subcutaneously in Sprague-Dawley rats and then extracted 4 days, 4 weeks, and 2 months post-implantation to examine the tissue response and histology. Representative results are shown in FIGS. 12A-12B, consistent for each type of implanted membrane. After 4 days, a very thin capsule (which fell apart upon gentle contact) formed around the membrane, and only a very minimal inflammatory response was observed. After 4 weeks and 2 months, a relatively thin, fatty capsule formed around the membrane and no obvious inflammatory response was observed.

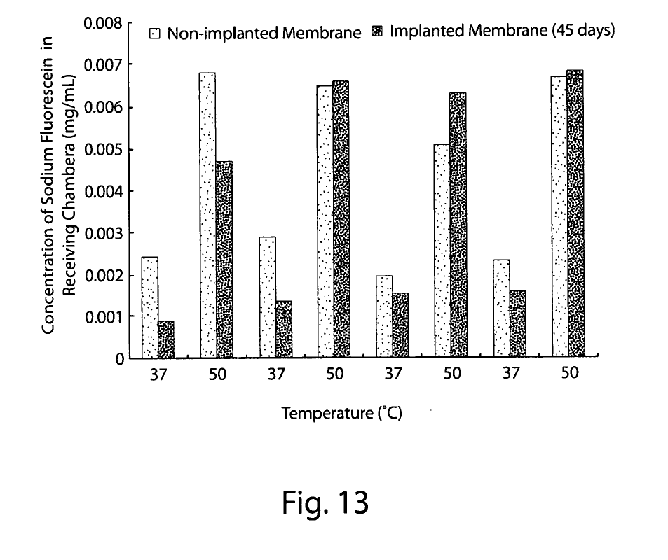

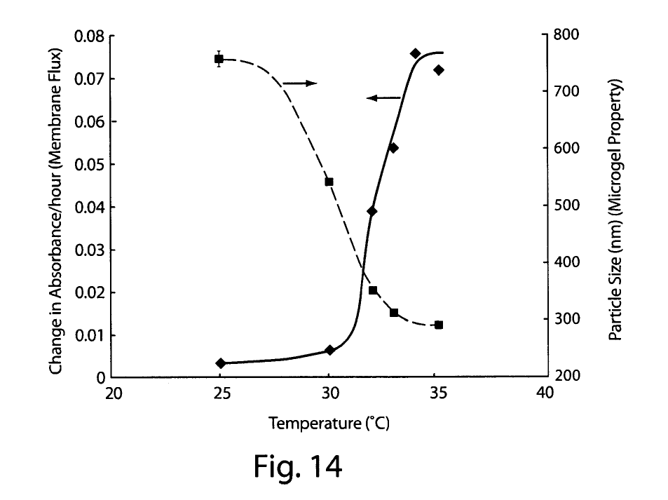

The membranes were also explanted and returned to the flow cell tests to determine if the flux amounts or the thermal flux differentials were impacted by implantation. The results are shown in FIG. 13 for a 30% ferrofluid-containing membrane extracted 45 days after implantation. Nearly identical flux results were observed before and after implantation, suggesting that protein fouling does not significantly impact the functionality of the device. Furthermore, as shown in FIG. 14, the implanted membrane maintained the sharp temperature sensitivity even after implantation, with the almost complete switching from the “off” state to the “on” state achieved between 38-42° C.

### EXAMPLE 3

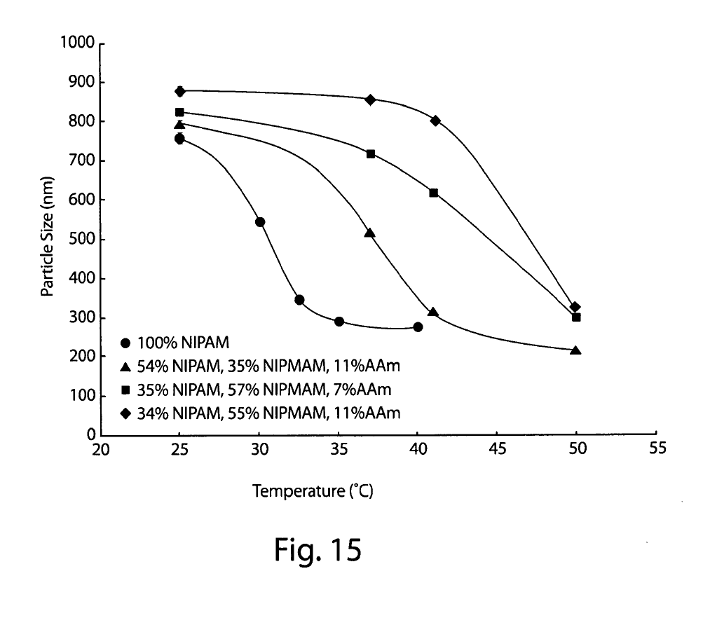

This example illustrates that the temperature at which a polymeric gel deswells (and thus the pores within the polymeric gel can be opened) can be tuned by copolymerizing other monomers with N-isopropylacrylamide (NIPAM). FIG. 15 shows the transition temperature behavior of NIPAM-based microgels prepared by copolymerizing N-isopropylmethacrylamide (NIPMAM, homopolymer transition temperature ˜42° C.) and acrylamide (AAm, homopolymer transition temperature >70° C.) with NIPAM. In particular, this figure shows particle size (in PBS) as a function of temperature for microgels prepared with different quantities of N-isopropylmethacrylamide (NIPMAM) and acrylamide (AAm). N-isopropylmethacrylamide increases the phase transition temperature by increasing the chain stiffness, while acrylamide is a significantly more hydrophilic than NIPAM.

NIPAM-only microgels have transition temperatures of ˜31° C. When 35% NIPMAM and 11% AAm are copolymerized into the gel, the transition temperature increases to ˜37° C. while 55% NIPMAM and 11% AAm results in a transition temperature of ˜46° C. Reducing the AAm content to 7% from 11% decreases the transition temperature to ˜42° C. In any case, the transition occurred over a relatively narrow temperature range (<5° C.). Based on these results, gels with a range of different transition temperatures can be easily be prepared. Practically, this may be applied in the invention to control the rates of drug release. For example, fabricating membranes containing a mix of gels with transition temperatures of ˜38° C. and ˜41° C. could be used to achieve multiple release rates using the same device, depending on the amount of heating applied to the device. Alternately, gels with higher transition temperatures which are only partially deswollen at the “on” temperature may be used to make the membrane less permeable to drug and thus facilitate lower rates of drug release. It should be noted that the total particle size change observed over the volume phase transition is similar regardless of the transition temperature of the gel used; as a result, the flux differential can be controlled independent of the transition temperature.

### EXAMPLE 4

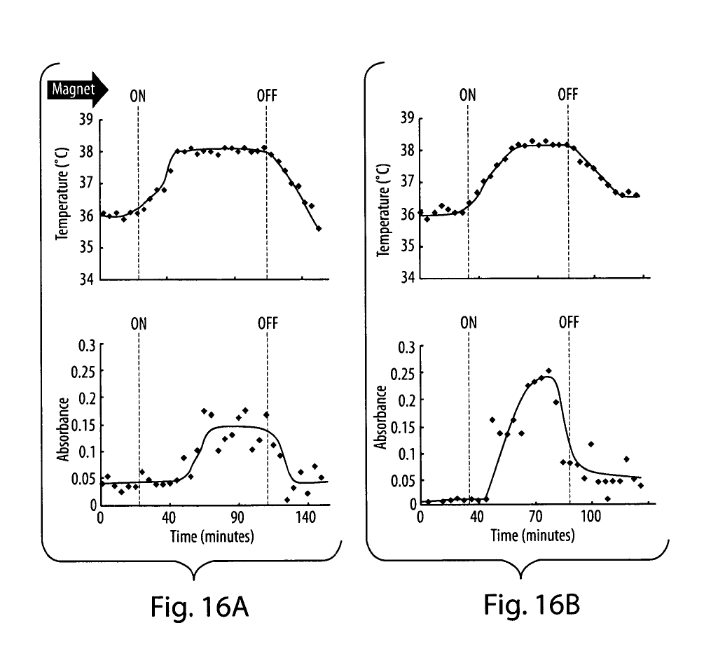

This example illustrates magnetically triggered release, using sodium fluorescein flux through a microgel/ferrofluid-loaded membranes, using a system similar to those previously described. See FIG. 16. Drug release rates (micrograms/min) were estimated by numerical integration of the areas under each of the concentration versus time curves.

FIG. 16A illustrates magnetically-induced flux using a 23% microgel, 19% ferrofluid membrane, while FIG. 16B illustrates the same for a 28% microgel, 19% ferrofluid membrane. Given identical loading of magnetite in both membranes, both membranes heated by ˜2.2° C. under an applied oscillating magnetic field. The membrane containing the lower microgel fraction (23 wt %) had a longer induction time the “on” state (˜35 minutes versus ˜15 minutes for the 28 wt % microgel membrane), returned to the baseline “off” flux level more slowly (˜15 minutes lag time versus <1 minute for the 28 wt % microgel membrane), and exhibited a lower average drug release in the “on” state relative to the “off” state (drug release rate =4.1 micrograms/min compared to 5.7 micrograms/min for the 28 wt % microgel membrane).

### EXAMPLE 5

To facilitate effective in vivo triggering, in this example, microgels were engineered to remain swollen (i.e. in the “off” state) at physiological temperature by copolymerizing N-isopropylacrylamide (NIPAM) with N-isopropylmethacrylamide (NIPMAM) and acrylamide (AAm). The methyl group of NIPMAM sterically inhibited the phase transition while AAm is more hydrophilic than NIPAM, both shifting the phase transition to higher temperatures. The ratio between the monomers was chosen to maximize the size change from the swollen to the collapsed state, in order to optimize membrane pore opening when triggered.

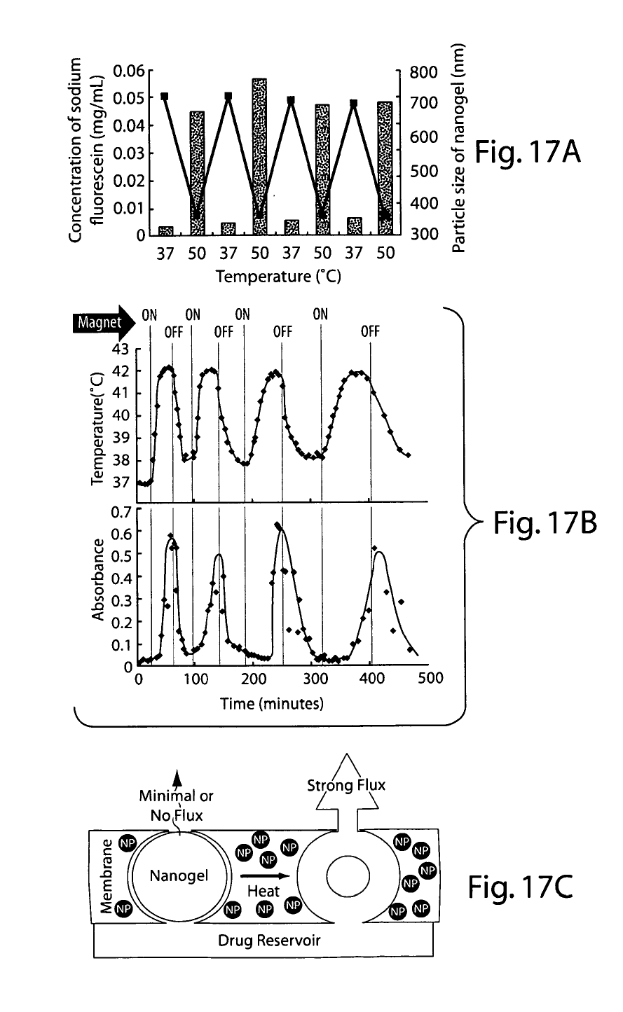

The ability of the membrane constituents and the composite membrane to trigger at physiologically relevant temperatures was evaluated using magnetic stimuli in this example. Microgels in free suspension in PBS underwent a ˜400 nm change in diameter upon heating from physiological temperature to 50° C. (FIG. 17A), with >90% of the total deswelling transition completed at 43° C. Thermal triggering of the microgel-containing membrane was tested by placing it between two chambers of a glass flow cell submerged in a water bath and evaluating the flux of sodium fluorescein across the membrane (i.e. between the chambers) as a function of time and temperature. A ˜20-fold higher flux of sodium fluorescein occurred at temperatures exceeding the volume phase transition temperature (˜40° C.) of the microgels (FIG. 17A). FT-IR analysis confirmed that this permeability enhancement coincided with a change in the hydrogen bonding within the membrane, consistent with the occurrence of a microgel volume phase transition. Furthermore, the fluorescein flux could be switched on and off over multiple thermal cycles with high reproducibility, suggesting that the microgel phase transition inside the membrane pores was fully reversible.

Magnetic triggering was evaluated in small-scale devices made by gluing two 1 cm diameter membrane disks to the ends of a 1 cm length of silicone tubing filled with a sodium fluorescein solution. The devices were mounted singly inside a semi-adiabatic flow cell in a solenoid coil, with constant water flow through the flow cell to permit continuous sampling of fluorescein release. FIG. 17B shows the magnetic triggering of the composite membrane. The magnetic nanoparticles embedded in the membrane heated inductively when subjected to an external oscillating magnetic field, heating which may be attributed to power absorption and subsequent magnetic relaxation of single-domain nanoparticles. At the applied magnetic frequency and field amplitude, the water inside the semi-adiabatic flow cell heated from 37° C. to ˜42° C. over the course of ˜10 minutes, at which point the temperature reached steady state. Heat generated by magnetite induction heating was transferred to the adjacent thermosensitive microgels, causing the microgels to shrink and permit drug diffusion out of the device. When the magnetic field was turned off, the device cooled, causing the microgels to re-swell and refill the membrane pores. As a result, the drug flux returned back to a near-zero value (FIG. 17C). As in the thermally-activated experiments, a 10-to-20-fold differential flux was observed between the “off” and “on” states. Furthermore, multiple on-off cycles could be performed without significantly changing the permeability of the membrane in the “off” state. This reproducibility suggests that magnetically-triggered physical distortion of the device plays no significant role in accelerating drug release from the membrane-based devices.

The membrane-based devices also permitted precise control of the amount of drug released as a function of the duration of the magnetic pulse. Table 1 shows the dose of fluorescein delivered for each of the four magnetically-activated cycles shown in FIG. 17, calculated by integrating the area under the absorbance vs. time curve for each cycle. The mass of compound released over each triggering cycle varied directly with the duration of the magnetic pulse (R²=0.995), with the rate of drug release varying by less than 10% in each cycle. Thus, drug release could be controlled by modulating both the frequency and duration of magnetic pulse. The devices turned “on” with only a 1-2 minute time lag after the solution temperature reached 40° C. and turn “off” with a ˜5-10 minute lag from the cooling temperature profile (FIG. 17B). This response rate was much more rapid than that seen with bulk, interpenetrating hydrogel networks, which can exhibit swelling kinetics on the order of hours.

TABLE 1

| | Duration of “on” | | Rate of drug |
|---|---|---|---|
| | cycle | Total mass | release |
| Cycle | (minutes) | released (mg) | (mg/min) |
| 1 | 35 | 0.43 | 0.012 |
| 2 | 40 | 0.47 | 0.012 |
| 3 | 57 | 0.69 | 0.012 |
| 4 | 75 | 0.83 | 0.011 |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of shall be closed or semi-closed transitional phrases, respectively, as set forth in the United States Patent Office Manual of Patent Examining Procedures, Section 2111.03.

What is claimed is:

1. An article containing a magnetically-susceptible material at least partially defining an enclosure containing a releasable species, wherein application of an oscillating magnetic field to the magnetically-susceptible material causes at least some release of the releasable species externally from the enclosure.

2. The article of claim 1, wherein the magnetically-susceptible material comprises iron.

3. The article of claim 1, wherein the article is implantable.

4. The article of claim 1, wherein the releasable species is contained in the magnetically-susceptible material.

5. The article of claim 1, wherein the article defines an enclosure containing the releasable species.

6. The article of claim 5, wherein the enclosure contains an aqueous solution containing the releasable species.

7. The article of claim 5, wherein the enclosure contains an organic solution containing the releasable species.

8. The article of claim 5, wherein the enclosure contains a solid containing the releasable species.

9. The article of claim 5, wherein the enclosure contains a gel containing the releasable species.

10. The article of claim 5, wherein the enclosure contains a liquid containing the releasable species.

11. The article of claim 1, wherein the releasable species is solid.

12. (canceled)

13. The article of claim 1, wherein the article further comprises a heat-sensitive material in thermal communication with the magnetically-susceptible material.

14. The article of claim 13, wherein the heat-sensitive material comprises a poly(N-alkyacrylamide).

15-16. (canceled)

17. The article of claim 1, wherein the article exhibits an increase of at least about 10% in the release of releasable species from the article, relative to the amount of release of the releasable species from the article in the absence of the oscillating magnetic field.

18. The article of claim 1, wherein heating the magnetically-susceptible material by at least about 0.5° C. causes the article to exhibit an increase of at least about 10% in the release of the releasable species from the article, relative to the amount of release of the releasable species from the article in the absence of heating.

19. The article of claim 1, wherein the releasable species comprises a drug.

20. The article of claim 1, wherein the enclosure is a particle.

21. The article of claim 1, wherein the enclosure is a liposome.

22. An article containing a magnetically-susceptible material at least partially defining an enclosure containing a releasable species, wherein application of an oscillating magnetic field to the magnetically-susceptible material causes an increase of release of the releasable species of at least about 10% from the implantable article, relative to the amount of release of the releasable species from the article in the absence of oscillating magnetic field.

23-25. (canceled)

26. An article comprising a magnetically-susceptible material at least partially defining an enclosure containing a releasable species, the magnetically-susceptible material being in thermal communication with a heat-sensitive material, wherein application of an oscillating magnetic field to the magnetically-susceptible material causes the heat-sensitive material to increase in temperature by at least about 0.5° C.

27-64. (canceled)
